(19) United States
(12) Patent Application Publication
(10) Pub. No.: US 2025/0243245 A1
(43) Pub. Date: Jul. 31, 2025

(54) Antibacterial Chimeric Peptides and Their Methods of Therapeutic Use

(71) Applicant: DiverseVitality.com LLC d/b/a/ Diverse Vitality, Santa Fe, NM (US)

(72) Inventor: Goutam Gupta

(21) Appl. No.: 18/855,851

(22) PCT Filed: Feb. 8, 2023
(86) PCT No.: PCT/US2023/062176

§ 371 Date: Oct. 10, 2024

Related U.S. Application Data
(60) Provisional application No. 63/330,173, filed on Apr. 12, 2022.

(51) Int. Cl. C07K 14/00; A61K 38/00

(52) U.S. Cl. CPC C07K 14/001; A61K 38/00

(57) ABSTRACT

The present invention includes the design and therapeutic application of novel α/β chimeric peptides formed by two different α/β segments coupled by a peptide linker. Through a combination of molecular modeling, bactericidal, and toxicity analyses the α/β chimeric peptides of the invention exhibit antibacterial effects in plants with low cytotoxicity in both plant and human cells.

### CROSS-REFERENCE TO RELATED APPLICATIONS

This International PCT application claims the benefit of and priority to U.S. Provisional Application No. 63/330,173, filed Apr. 12, 2022. The entire specification, claims, and figures of the above-referenced application is hereby incorporated, in its entirety by reference.

### STATEMENT OF GOVERNMENT INTEREST

This invention was made with government support under National Institute of Food and Agriculture (NIFA) Grant No. 2020-70029-33199. The government retains certain rights in this invention.

### SEQUENCE LISTING

The instant application contains contents of the electronic sequence listing (90325-00021-Sequence-Listing.xml; Size: 50,658 bytes; and Date of Creation: Feb. 8, 2023) is herein incorporated by reference in its entirety.

### TECHNICAL FIELD

The inventive technology includes systems, methods, and compositions for the design, production, and use of novel antibacterial compounds. The invention may specifically include novel engineered antimicrobial peptide compositions and their methods of use in treating bacterial infections in plants.

### BACKGROUND

Fruit crops provide both fresh and processed fruits to the consumers for health and nourishment. However, growers of these foods face serious threats from chronic and emerging bacterial diseases. Effective tools are urgently needed for the treatment of bacterial diseases in fruit crops, such as citrus, grape and apple crops. For example, of all citrus diseases, Huanglongbing (HLB) is the most devastating one caused by Candidatus Liberibacter asiaticus CLas or Liberibacter), a gram-negative bacterium, which is transmitted by Asian citrus psyllids (ACP). Since the first detection in 2005, the Florida citrus industry has witnessed a decline in production by 80% in sweet orange and 90% in grapefruit. In Texas, HLB was first detected in 2012 and is currently confirmed in 28% commercial and 40% residential groves. In California, so far, only 3,053 isolated HLB cases have been identified and removed as mandated by the state. Although, the major citrus-producing states experience different HLB pressures, all of them need effective, safe, and affordable tools to treat and prevent HLB. The current disease management tools consist of insecticide spray to limit the spread of psyllids. Also, nutrients, chemicals, and antibiotics have been applied for disease management. Unfortunately, none of the current disease management tools provide cure for HLB.

In another example, fire blight, like HLB, is a destructive disease that mainly attacks apples and pears. It is caused by the bacterium Erwinia amylovora, which infects blossoms, fruits, vegetative shoots, woody tissues, and rootstock crowns. It is estimated that the U.S. apple producers suffer an average annual loss of $100 million due to fire blight. The diversity of host tissues susceptible to infection, combined with the limited number of available and effective disease management tools, has made it difficult to stop or slow the progress of fire blight epidemics.

Almost a decade ago, the present inventors introduced the concept of host-based therapy for the treatment and prevention of bacterial diseases in human and plant. The application of this concept was successful in countering diseases caused by intact bacteria or their toxins secreted by them. This strategy was primarily focused on enhancing the host innate immunity, which is the first line of defense against the invading pathogens. Notably, plant's innate immunity repertoire contains pathogenesis-related (PR) or defense proteins to clear the pathogens or block pathogenesis. However, evolution of bacterial resistance often renders the PR proteins ineffective. Therefore, one aim of the current invention is to introduce sequence/structure modifications in the PR proteins to help them overcome bacterial resistance while showing high activity against the invading pathogens.

One of the successful applications of this strategy described by Gupta et al., involved the design of helix-turn-helix (HTH) peptides and their use in the treatment two bacterial diseases, namely Pierce's Disease (PD) in grape caused by xylem-limited Xylella fastidiosa (Xf) and HLB in citrus caused by phloem-limited CLas. These HTH peptides were designed by joining two identical helical amphipathic peptides by a sharp type-II GPGR turn. While each helix had homologous segments in grape and citrus proteins, the whole length of the artificially constructed HTH peptide showed little homology with any grape or citrus protein segment. Toxicity assays verified that the HTH peptides showed no toxicity to plant leaves or human cells at the dose they showed bactericidal activity. In field trials, two HTH peptides (code names: 28P-2 and 36P-1) showed efficacy for the treatment of PD in grapevines and HLB in citrus. Laboratory studies revealed that 36P-1 was 14-times more active on CLas than 28P-2. But 36P-1 appeared to be toxic at the treated dose, which required design of 36P-1 analogs with similar activity but with very low or no toxicity.

This prompted the present inventors to develop novel strategies to overcome the practical shortcoming demonstrated in the prior art above by combining two different peptide segments rather than two identical ones. For example, as shown below, The present inventors have shown that plant proteins contain α or β peptide segments with antibacterial activities. Again, these peptide segments were selected from the proteins belonging to the plant innate immune repertoire. The bactericidal peptide segments (designated here as single units) in the host proteins were discovered almost three decades ago. They were shown to be active against antibiotic-resistant planktonic and biofilm bacteria. In addition, they were expected to exert immune stimulatory activity. However, it was soon discovered bacteria quickly evolve resistance against the host peptide by modifying their membrane structure limit this peptide-based antibacterial strategy.

To overcome these limitations, the present inventors sought to combine two antibacterial peptide segments as described herein to overcome bacterial resistance and retain bactericidal effect at the same time. Moreover, the novel chimeric peptides described by the present invention are not toxic to humans and plants and are stimulatory to host immune system.

### SUMMARY OF THE INVENTION

In one aspect, the inventive technology described herein include the design strategy and therapeutic application of novel chimeric peptides. In a preferred embodiment, the novel chimeric peptides of the invention include a combination of two different antibacterial peptides in a chimeric peptide resulting in enhanced bactericidal activity on and clearance of the invading pathogen, but also augmentation of plant immunity during infection.

One aspect of the inventive technology may include a novel antimicrobial peptide comprising a first α/β peptide domain and a second α/β peptide domain coupled by a linker domain forming an antimicrobial chimeric peptide sequence, wherein the first and second α/β peptide domain are derived from the host plant, and wherein each domain is a distinct antimicrobial peptide. The antimicrobial peptides of the invention exhibit: 1) increased bactericidal effects compared to a single endogenous antimicrobial peptides; 2) increased efficiency of attachment and/or insertion into a bacterial membrane compared to a single endogenous antimicrobial peptide; 3) lower susceptibility to bacterial resistance compared to a single endogenous antimicrobial peptide; and 4) low or no toxicity to mammalian cells; 5) low or no phytotoxicity to plant and human cells; 6) increase the plant's innate immune response.

Additional aspects of the inventive technology may include novel antimicrobial peptides having a first α/β peptide domain and a second α/β peptide domain coupled by a linker domain forming an antimicrobial chimeric peptide sequence, wherein the first and second α/β peptide domains are derived from endogenous antimicrobial peptides from a plant, and preferably a fruit plant, such as a citrus, grape, and/or apple plants among others.

Additional aspects of the inventive technology may include novel antimicrobial peptides having a first α/β peptide domain and a second α/β peptide domain coupled by a linker domain forming an antimicrobial chimeric peptide sequence, wherein the first α/β peptide domain and the second α/β peptide domain are selected from the group consisting of: a unit A peptide, a unit C/B peptide, a unit D peptide, and a unit E peptide as defined herein. In a preferred aspect, first α/β peptide domain comprises a unit A peptide and said second α/β peptide domain is a C/B peptide, or a unit D peptide as defined herein.

Additional aspects of the inventive technology may include novel antimicrobial peptides having a first α/β peptide domain and a second α/β peptide domain coupled by a linker domain forming an antimicrobial chimeric peptide sequence, wherein the first α/β peptide domain and the second α/β peptide domain are selected from the group consisting of: SEQ ID NO.'s 1-6, and/or 57, or a variant or homolog thereof.

Additional aspects of the inventive technology may include novel antimicrobial peptides having a first α/β peptide domain and a second α/β peptide domain, coupled by a linker domain, according to SEQ ID NO.'s 21-45, and 54. Additional aspects of the inventive technology may include novel antimicrobial peptides having a first α/β peptide domain and a second α/β peptide domain coupled by a linker domain selected from: SEQ ID NO.'s 25, 31-33, 45 and 54.

Additional aspects of the inventive technology may include novel antimicrobial peptides having a first α/β peptide domain and a second α/β peptide domain coupled by a linker domain selected from: SEQ ID NO.'s 7-16, and/or 58. Additional aspects of the inventive technology may include novel antimicrobial peptides having a first α/β peptide domain and a second α/β peptide domain coupled by a linker domain selected from: SEQ ID NO.'s 25, 31-33, 45 and 54.

Additional aspects of the inventive technology may include embodiments wherein an antimicrobial peptide of the invention is encoded by a polynucleotide comprising a nucleic acid sequence. Additional aspects of the inventive technology may include embodiments wherein the antimicrobial peptide of the invention is encoded by a polynucleotide which is further linked to a promoter to produce an expression vector.

Additional aspects of the inventive technology may include embodiments wherein the antimicrobial peptide is encoded by a polynucleotide operably linked to a promotor, and wherein a plant or plant cell heterologously express the antimicrobial peptide. In a preferred aspect, such a plant or plant cell may include a citrus plant or citrus plant cell, or more preferably a plant infected with, or at risk of being infected with Huanglongbing (HLB), Fire blight, or Pierce's disease (PD).

Additional aspects of the inventive technology may include embodiments wherein for the antimicrobial peptide of the invention may be used as a therapeutic agent for plants infected with and/or at risk of being infected by a bacterial pathogen, an in particular a gram-negative bacterial pathogen Huanglongbing (HLB), Fire blight, or Pierce's disease. Additional aspects of the inventive technology may include embodiments wherein the antimicrobial peptide may be used as a therapeutic agent for plants infected with and/or at risk of being infected by Erwinia amylovora, Candidatus Liberibacte asiaticus ((Las), and Xylella fastidiosa (Xf).

Additional aspects of the inventive technology may include embodiments wherein the antimicrobial peptide may be topically applied to infected with, or at risk of being infected with Huanglongbing (HLB), Fire blight, or Pierce's disease.

Additional aspects of the inventive technology may include embodiments wherein the antimicrobial peptide may be used as a therapeutic agent for the treatment and/or prevention of Huanglongbing (HLB) in plants, and preferably citrus plants. Alternative embodiments include wherein the antimicrobial peptide may be used as a therapeutic agent for the treatment and/or prevention of Huanglongbing (HLB) in plants, wherein the antimicrobial peptide increase the immunity, and preferably innate immunity of the plant as a method treating HLB.

Additional aspects of the inventive technology may include embodiments wherein the antimicrobial peptide may be used as a therapeutic agent for the treatment and/or prevention of Fire blight in plants, and preferably apple plants. Alternative embodiments include wherein the antimicrobial peptide may be used as a therapeutic agent for the treatment and/or prevention of Fire blight in plants, wherein the antimicrobial peptide increase the immunity, and preferably innate immunity of the plant as a method treating Fire blight.

Additional aspects of the inventive technology may include embodiments wherein the antimicrobial peptide may be used as a therapeutic agent for the treatment and/or prevention of Pierce's disease in plants, and preferably grape plants. Alternative embodiments include wherein the antimicrobial peptide may be used as a therapeutic agent for the treatment and/or prevention of Pierce's disease in plants, wherein the antimicrobial peptide increase the immunity, and preferably innate immunity of the plant as a method treating Pierce's disease.

Additional aspects include the embodiments shown in the claims of U.S. Provisional Application No. 63/330,173, filed Apr. 12, 2022, which are incorporated herein by reference.

Additional aspects of the inventive technology will be evident from the detailed description and figures presented below.

### BRIEF DESCRIPTION OF DRAWINGS

FIG. 1. Bioluminescence data show the viability of E. coli BL21 at different peptide doses for the α/β chimeras (A) UGK-13, (B) UGK-17, and (C) 30P-3; also shown are the dose response curves for the single unit constituents of the chimeras, i.e., 11P-1 and 11P-3. (D) The dose response curve for L19GA with two b-strands stabilized by 2 S—S bridges. E. coli BL21 (5×10⁵ cfu) were incubated with peptide for 24 hours.

FIG. 2. Two-color fluorescence assay after 1 hour peptide incubation of E. coli BL21 (5×10⁵ cfu). (A) % live cells by treatment of different peptides at 20 mM concentration. (B) fluorescent images of live and dead bacteria for the same experiment of (A).

FIG. 3. Monitoring toxicity after infiltration of different peptides at 15-25 mM concentration for 24 and 96 hours. (A) Tomato leaves, top and tobacco leaves for 24 hours. (B) Tomato leaves, top and tobacco leaves for 96 hours.

FIG. 4. Cell viability due to treatment of different peptides at 20 mM concentration by (A) hemolytic and (B) MTT assay.

FIG. 5. Cartoon diagrams (blue to red from N to C terminal) of different peptides. (A) 30P-3, (B) UGK-13, (C) UGK-17, (D) UGK-9, and (E) L19GA.

FIG. 6. Bacterial clearance of (A, B) CLas by DNA and RNA qPCR and (C, D) E. amylovora by DNA and RNA qPCR.

FIG. 7A. The selected genes encode: Lipid-transfer protein 2, LTP2 (XM_006482145.3), Ethylene-responsive transcription factor 3, ERF003 (XM_006483296.3), Chitinase (XM_015532796.2), Zinc finger, C2H2 type (XM_015531045.2), GDSL esterase (XM_006478917.3), Abscisic acid induced-regulated protein (XM_025101123.1), LEA protein5, LEA5 (NM_001289140.1), Cytochrome P450 82G1 (XM_006479159.3), sodium/hydrogen exchanger 2 (XM_006479811.3), Phloem-specific lectin PP2-like protein (XM_025095878.1), Ethylene-responsive transcription factor 6, ERF006 (XM_006466962.3), Sweet sugar transporter 3 (XM_006490501.3), MAPK6 (XM_025097223.1), Defensein (XM_006470821.3), EDS (XM_006476627.2), Col1 (XM_006486308.3), MAPKK3 (XM_006470193.3), WRKY24 (XM_006468068.3), WRKY4 (XM_006483024.2), PR1 (XM_006474081.3), MYB13 (XM_006479482.2), PR2 (KAH9738797.1), PR3 (KDO71433.1). The selected genes were also shown by RNA-seq to be differentially expressed upon CLas infection in citrus. The expression of the selected genes in treated and untreated citrus leaves was normalized relative to the expression of the housekeeping GAPDH.

FIG. 7B. The selected genes encode: Jaz17, JA receptor (MDP0000241358), bHLH, JA induced transcription factor (MDP0000242554), EBP, ethylene induced binding GCC element binding transcription factor (MDP0000241358), AP2/ERF: regulates the biosynthesis of carotenoid by regulating the transcription of PSY, PAL1, SA-inducing PHE ammonia lyase 1 (MDP0000388769) Chalcone and stilbene synthase in flavonoid synthesis (MDP0000168735), ribonuclease-like PR (MDP0000782085), Apple defensin (MDP0000362305), Acidic endochitinase-like protein (MDP0000280265), Intracellular Ras-group-related LRR protein (MDP0000281307), Chlorophyl binding protein PSII LHC (MDP0000708928), The light-harvesting complex, LHC (MDP0000601491), NAD(P)H dehydrogenase (MDP0000509613), Peroxidase super family (MDP243237), Sorbitol dehydrogenase, SDH-GroES-like zinc-binding alcohol dehydrogenase family protein (MDP0000515106), MDP0000850409, MDP0000364657, Gibberellic acid stimulated Arabidopsis (GASA) gene (MDP0000201700), Xyloglucan endotransglucosylases/hydrolases, XTH (MDP0000361876), Hydroxyproline-rich glycoprotein family protein (MDP0000248516), Fasciclin-like arabinogalactan-protein 7 (MDC015146.108:31720-32772), MDP0000297541, proline-rich receptor-like protein kinase (MDP0000511014), MDP0000268505, Nicotianamine synthase-like 4 (MDP0000412490), Hydroxyproline-rich glycoprotein family protein: MDP0000248516. The selected genes were also shown by RNA-seq to be differentially expressed upon E. Amylovora infection in APPLE. The expression of the selected genes in treated and untreated apple leaves was normalized relative to the expression of the housekeeping GAPDH.

FIGS. 8A-H tobacco and tomato leaves treated with peptides, UGK-13, UGK-17, UGK-9, and 30P-3 for all 24, 48, 72, and 96 hours of infiltration. Note that like the control, the peptide infiltrated tomato and tobacco leaves showed no corrosion beyond the infiltrated spots.

FIG. 9 example peptide cartoon diagrams of candidates α/βP1-6 in one embodiment thereof.

FIG. 10 clearance of CLas (A-B) and (C-D) A. amylovora by candidates α/βP1-6 in one embodiment thereof.

FIGS. 11A-B shows heatmap expression analysis by qPCR of innate immune genes after treatment with different candidates α/βP1-6.

FIG. 12 show treated and untreated samples and % clearance of infected samples at 25 and 70 days.

### DETAILED DESCRIPTION OF INVENTION

Disclosed herein are novel systems, methods, and compositions for the treatment of bacterial infections in plants. These inventions may further include novel systems, methods, and compositions for the treatment of gram-negative bacterial infections in plants. In one specific embodiment, the invention may include novel systems, methods, and compositions for the treatment of HLB disease caused by CLas in plants. In another specific embodiment, the invention may include novel systems, methods, and compositions for the treatment of fire blight disease caused by Erwinia amylovora. In another specific embodiment, the invention may include novel systems, methods, and compositions for the treatment of Pierce's disease caused by Xylella fastidiosa (Xf). In these preferred embodiments, the invention may include novel antimicrobial peptides that may be used to treat susceptible or already infected plants, which may cure, or lower the bacterial load and increase the productive years of the a plant infected with, or at risk of being infected with Huanglongbing (HLB), Fire blight, or Pierce's disease.

Additional embodiments of the invention may include the generation of transgenic plants expressing one or more of the antimicrobial chimeric peptides of the invention that provide resistance against infection by gram-negative bacterial pathogens. Additional embodiments of the invention may include the generation of transgenic HLB-resistant citrus plants that express one or more of the antimicrobial chimeric peptides of the invention. Further embodiments of the invention may include the generation of transgenic fire blight-resistant plants that express one or more of the antimicrobial chimeric peptides of the invention. Further embodiments of the invention may include the generation of transgenic Xf-resistant plants that express one or more of the antimicrobial chimeric peptides of the invention.

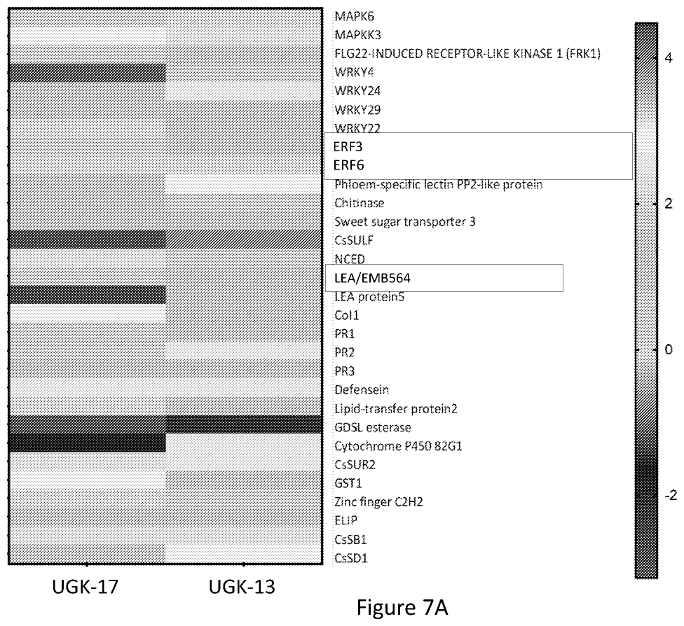

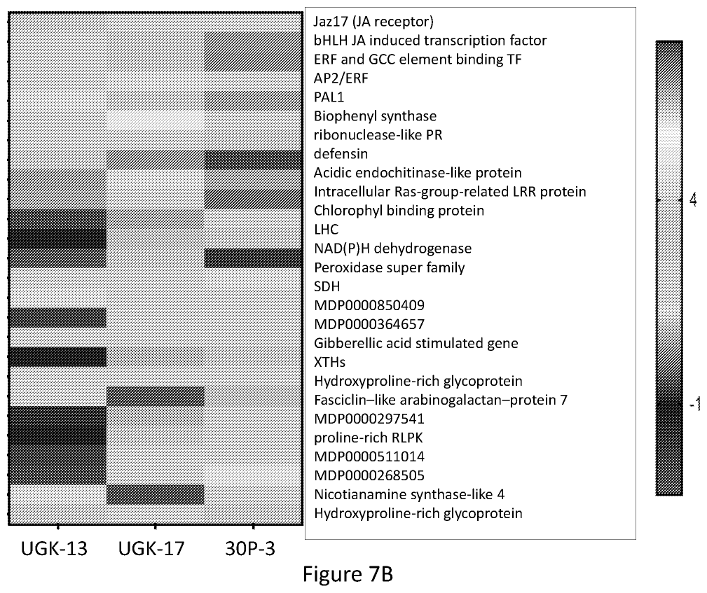

Additional embodiments of the invention may include the generation of transgenic plants expressing one or more of the antimicrobial chimeric peptides of the invention that increase the plant's innate immune response, preferably though the regulation of one or more genes associated with the plant's innate immune response. Exemplary innate immune response associated genes are provided in FIG. 7A-B, and their sequences are incorporated herein by reference.

In one embodiment, the present invention includes the design and therapeutic application of novel α/β chimeric peptides formed by two different α/β segments. As shown herein, through a combination of molecular modeling, bactericidal, and toxicity analyses the α/β chimeric peptides of the invention exhibit antibacterial effects in plants with low cytotoxicity. In one preferred embodiment, novel α/β chimeric peptides UGK-13 and UGK-17 exhibit bacterial effects in plants with low cytotoxicity, and specifically clear E. amylovora from infected apple leaves with fire blight. In another embodiment shown herein, the novel α/β chimeric peptide UGK-17 clears CLas from infected citrus with HLB. The present inventors further show by qPCR that both UGK-13 and UGK-17 upregulate select gene expression augmenting the plant host's innate immunity in during infection.

In another embodiment, the present invention describes methods and compositions for the rational design of novel α/β chimeric peptides, in addition to their therapeutic use in treating plant pathogens, such as HLB, fire blight and Pierce's disease. This general strategy is demonstrated here by designing plant derived chimeric peptides and by testing their efficacy against HLB in citrus and fire blight in apple, as well as Pierce's disease in grapes. These chimeric peptides of the invention are constructed by joining two different segments from citrus/apple or grape proteins. These segments in isolation show (or are rationally predicted to show) activity against gram-negative bacteria by lysing their membranes. Typically, the individual segments show low activity; however, when present in a chimera scaffold, their antibacterial activity is increase due to their synergetic action. These segments are generally unstructured in isolation. However, when they encounter the bacterial membrane, the α/β chimera scaffold may facilitate the formation of α or β structures even without the association of a bacterial membrane.

As noted above, plants and other organisms may generate a variety of endogenous single antimicrobial peptides to defend against bacterial infections. As such, in one embodiment, the invention may include an antimicrobial chimeric peptide having a first α/β peptide domain and a second α/β peptide domain coupled by a linker domain forming a α/β scaffold formation where each peptide domain may be selected from the group of antimicrobial peptides that may be endogenous to a host plant, such as a citrus plant. In one embodiment, a first α/β peptide domain and a second α/β peptide domain may each be selected from the group of peptides consisting of a unit A peptide, a unit C/B peptide, a unit D peptide, and a unit E peptide. In a preferred embodiment, first α/β peptide domain may comprise a unit A peptide and said second α/β peptide domain may include a C/B peptide, or a unit D peptide. In another embodiment, a first α/β peptide domain and a second α/β peptide domain may each be selected from the group of peptides consisting of: SEQ ID NO.'s 1-6, and/or 57, or a variant or homolog thereof. (Notably, as described below, the disclosure of any SEQ ID NO, specifically incorporates all variant and homologs of the same.)

As such, in one embodiment, the invention may include an antimicrobial chimeric peptide having a first α/β peptide domain and a second α/β peptide domain coupled by a linker domain wherein the chimeric peptide sequence is selected from the group consisting of: SEQ ID NO.'s 21-45, and 54. In another embodiment, the invention may include an antimicrobial chimeric peptide having a first α/β peptide domain and a second α/β peptide domain coupled by a linker domain wherein the chimeric peptide sequence is selected from the group consisting of: SEQ ID NO.'s 25, 31-33, 45 and 54, or a variant or homolog thereof. In another embodiment, the invention may include an antimicrobial chimeric peptide having a first α/β peptide domain and a second α/β peptide domain coupled by a linker domain wherein the chimeric peptide sequence is selected from the group consisting of: SEQ ID NO.'s 25, 31-33, 45 and 54, or a variant or homolog thereof.

As noted above, a linker domain may couple together a first and second peptide domain. In one embodiment, this linker domain may include an amino acid sequence according to SEQ ID NO. 7-16, and/or 58.

As noted above, the invention may include one or more of the antimicrobial peptides identified herein to treat bacterial infections in plants. For example, the invention may include one or more of the antimicrobial peptides described herein as a therapeutic agent for plants infected with and/or at risk of being infected by a bacterial pathogen, preferably a gram-negative bacterial pathogen. In this embodiment, one or more of the antimicrobial peptides identified herein may be used a therapeutic agent for plants infected with and/or at risk of being infected by Erwinia amylovora, Candidatus Liberibacte asiaticus ((Las), and Xylella fastidiosa (Xf), the causative agents of Fire Blight, HLB, and Pierce's disease, respectively.

In one specific example, an antimicrobial peptide, identified as amino acid SEQ ID NO. 32, or a variant or homolog of the same, may include a first α/β peptide domain having a peptide according to SEQ ID NO. 46, and said second α/β peptide domain having a peptide according to SEQ ID NO. 47, coupled by a linker domain according SEQ. ID NO. 12, forming a α/β scaffold formation. This engineered peptide may be a increase the plant's innate immune response plants by regulating one or more genes responsible for the plant's innate immunity (See FIG. 7). In this embodiment, such engineered peptide identified as UGK-13 (SEQ ID NO. 32) may exhibit a therapeutically effective resistance to bacterial or other pathogens through an enhanced innate immune response as compared to a single endogenous antimicrobial peptide sub-component (SEQ ID NOs 46-47), and in particular in citrus, grape and apple plants.

In one specific example, an antimicrobial peptide, identified as amino acid SEQ ID NO. 32, or a variant or homolog of the same, may include a first α/β peptide domain having a peptide according to SEQ ID NO. 46, and said second α/β peptide domain having a peptide according to SEQ ID NO. 47, coupled by a linker domain according SEQ. ID NO. 12, forming a α/β scaffold formation. This engineered antimicrobial peptide may be a therapeutic agent for plants, and more specifically citrus plants infected with and/or at risk of being infected by Candidatus Liberibacte asiaticus ((Las). In this embodiment, such engineered antimicrobial peptide identified as UGK-13 (SEQ ID NO. 32) may exhibit a therapeutic effect against Candidatus Liberibacte asiaticus (CLas), or other gram-negative bacteria through an enhanced bactericidal effect as compared to a single endogenous antimicrobial peptide sub-component (SEQ ID NOs 46-47) in apple plants exhibiting Huanglongbing (HLB).

In one specific example, an antimicrobial peptide, identified as amino acid SEQ ID NO. 32, or a variant or homolog of the same, may include a first α/β peptide domain having a peptide according to SEQ ID NO. 46, and said second α/β peptide domain having a peptide according to SEQ ID NO. 47, coupled by a linker domain according SEQ. ID NO. 12, forming a α/β scaffold formation. This engineered antimicrobial peptide may be a therapeutic agent for plants, and more specifically citrus plants infected with and/or at risk of being infected by E. amylovora. In this embodiment, such engineered antimicrobial peptide identified as UGK-13 (SEQ ID NO. 32) may exhibit a therapeutic effect against E. amylovora, or other gram-negative bacteria through an enhanced bactericidal effect as compared to a single endogenous antimicrobial peptide sub-component (SEQ ID NOs 46-47) in apple plants exhibiting Fire Blight.

In one specific example, an antimicrobial peptide, identified as amino acid SEQ ID NO. 32, or a variant or homolog of the same, may include a first α/β peptide domain having a peptide according to SEQ ID NO. 46, and said second α/β peptide domain having a peptide according to SEQ ID NO. 47, coupled by a linker domain according SEQ. ID NO. 12, forming a α/β scaffold formation. This engineered antimicrobial peptide may be a therapeutic agent for plants, and more specifically citrus plants infected with and/or at risk of being infected by Xylella fastidiosa (Xf). In this embodiment, such engineered antimicrobial peptide identified as UGK-13 (SEQ ID NO. 32) may exhibit a therapeutic effect against Xylella fastidiosa (Xf), or other gram-negative bacteria through an enhanced bactericidal effect as compared to a single endogenous antimicrobial peptide sub-component (SEQ ID NOs 46-47) in apple plants exhibiting in grape plants exhibiting Pierce's disease.

In one specific example, an antimicrobial peptide, identified as amino acid SEQ ID NO. 31, or a variant or homolog of the same, may include a first α/β peptide domain having a peptide according to SEQ ID NO. 5, and said second α/β peptide domain having a peptide according to SEQ ID NO. 50, coupled by a linker domain according SEQ. ID NO. 11, forming a α/β scaffold formation. This engineered antimicrobial peptide may be a therapeutic agent for plants, and more specifically citrus plants infected with and/or at risk of being infected by Candidatus Liberibacte asiaticus ((Las). In this embodiment, such engineered antimicrobial peptide identified as SEQ ID NO. 31 may exhibit a therapeutic effect against Candidatus Liberibacte asiaticus ((Las), or other gram-negative bacteria through an enhanced bactericidal effect as compared to a single endogenous antimicrobial peptide sub-component (SEQ ID NOs 5-50) in apple plants exhibiting Huanglongbing (HLB).

In one specific example, an antimicrobial peptide, identified as amino acid SEQ ID NO. 31, or a variant or homolog of the same, may include a first α/β peptide domain having a peptide according to SEQ ID NO. 5, and said second α/β peptide domain having a peptide according to SEQ ID NO. 50, coupled by a linker domain according SEQ. ID NO. 11, forming a α/β scaffold formation. This engineered antimicrobial peptide may be a therapeutic agent for plants, and more specifically citrus plants infected with and/or at risk of being infected by E. amylovora. In this embodiment, such engineered antimicrobial peptide identified as UGK-13 (SEQ ID NO. 31) may exhibit a therapeutic effect against E. amylovora, or other gram-negative bacteria through an enhanced bactericidal effect as compared to a single endogenous antimicrobial peptide sub-component (SEQ ID NOs 5-50) in apple plants exhibiting Fire Blight.

In one specific example, an antimicrobial peptide, identified as amino acid SEQ ID NO. 31, or a variant or homolog of the same, may include a first α/β peptide domain having a peptide according to SEQ ID NO. 5, and said second α/β peptide domain having a peptide according to SEQ ID NO. 50, coupled by a linker domain according SEQ. ID NO. 11, forming a α/β scaffold formation. This engineered antimicrobial peptide may be a therapeutic agent for plants, and more specifically citrus plants infected with and/or at risk of being infected by Xylella fastidiosa (Xf). In this embodiment, such engineered antimicrobial peptide identified as SEQ ID NO. 31 may exhibit a therapeutic effect against Xylella fastidiosa (Xf), or other gram-negative bacteria through an enhanced bactericidal effect as compared to a single endogenous antimicrobial peptide sub-component (SEQ ID NOs 5-50) in apple plants exhibiting in grape plants exhibiting Pierce's disease.

In another specific example, an antimicrobial peptide, identified as amino acid SEQ ID NO. 33, or a variant or homolog of the same, may include a first α/β peptide domain having a peptide according to SEQ ID NO. 48, and said second α/β peptide domain having a peptide according to SEQ ID NO. 49, coupled by a linker domain according SEQ. ID NO. 12, forming a α/β scaffold formation. This engineered antimicrobial peptide may be a therapeutic agent for plants, and more specifically citrus plants infected with and/or at risk of being infected by F. amylovora. In this embodiment, such engineered antimicrobial peptide identified as UGK-17 (SEQ ID NO. 33) may exhibit a therapeutic effect against E. amylovora, or other gram-negative bacteria through an enhanced bactericidal effect as compared to a single endogenous antimicrobial peptide sub-component (SEQ ID NOs 48-49) in apple plants exhibiting Fire Blight.

In another specific example, an antimicrobial peptide, identified as amino acid SEQ ID NO. 33, or a variant or homolog of the same, may include a first α/β peptide domain having a peptide according to SEQ ID NO. 48, and said second α/β peptide domain having a peptide according to SEQ ID NO. 49, coupled by a linker domain according SEQ. ID NO. 12, forming a α/β scaffold formation. This engineered antimicrobial peptide may be a therapeutic agent for plants, and more specifically citrus plants infected with and/or at risk of being infected by Candidatus Liberibacte asiaticus ((Las). In this embodiment, such engineered antimicrobial peptide identified as UGK-17 (SEQ ID NO. 33) may exhibit a therapeutic effect against Candidatus Liberibacte asiaticus ((Las), or other gram-negative bacteria through an enhanced bactericidal effect as compared to a single endogenous antimicrobial peptide sub-component (SEQ ID NOs 48-49) in apple plants exhibiting Huanglongbing (HLB).

In one specific example, an antimicrobial peptide, identified as amino acid SEQ ID NO. 33, or a variant or homolog of the same, may include a first α/β peptide domain having a peptide according to SEQ ID NO. 48, and said second α/β peptide domain having a peptide according to SEQ ID NO. 49, coupled by a linker domain according SEQ. ID NO. 12, forming a α/β scaffold formation. This engineered peptide may be a increase the plant's innate immune response plants by regulating one or more genes responsible for the plant's innate immunity (See FIG. 7). In this embodiment, such engineered peptide identified as UGK-17 (SEQ ID NO. 33) may exhibit a therapeutically effective resistance to bacterial or other pathogens through an enhanced innate immune response as compared to a single endogenous antimicrobial peptide sub-component (SEQ ID NOs 48-49), and in particular in citrus, grape and apple plants.

In one specific example, an antimicrobial peptide, identified as amino acid SEQ ID NO. 31, or a variant or homolog of the same, may include a first α/β peptide domain having a peptide according to SEQ ID NO. 46, and said second α/β peptide domain having a peptide according to SEQ ID NO. 47, coupled by a linker domain according SEQ. ID NO. 12, forming a α/β scaffold formation. This engineered antimicrobial peptide may be a therapeutic agent for plants, and more specifically citrus plants infected with and/or at risk of being infected by Candidatus Liberibacte asiaticus (CLas). In this embodiment, such engineered antimicrobial peptide identified as SEQ ID NO. 31 may exhibit a therapeutic effect against Candidatus Liberibacte asiaticus ((Las), or other gram-negative bacteria through an enhanced bactericidal effect as compared to a single endogenous antimicrobial peptide sub-component (SEQ ID NOs 46-47) in apple plants exhibiting Huanglongbing (HLB).

In one specific example, an antimicrobial peptide, identified as amino acid SEQ ID NO. 31, or a variant or homolog of the same, may include a first α/β peptide domain having a peptide according to SEQ ID NO. 46, and said second α/β peptide domain having a peptide according to SEQ ID NO. 47, coupled by a linker domain according SEQ. ID NO. 12, forming a α/β scaffold formation. This engineered antimicrobial peptide may be a therapeutic agent for plants, and more specifically citrus plants infected with and/or at risk of being infected by E. amylovora. In this embodiment, such engineered antimicrobial peptide identified as UGK-13 (SEQ ID NO. 31) may exhibit a therapeutic effect against E. amylovora, or other gram-negative bacteria through an enhanced bactericidal effect as compared to a single endogenous antimicrobial peptide sub-component (SEQ ID NOs 46-47) in apple plants exhibiting Fire Blight.

In one specific example, an antimicrobial peptide, identified as amino acid SEQ ID NO. 31, or a variant or homolog of the same, may include a first α/β peptide domain having a peptide according to SEQ ID NO. 46, and said second α/β peptide domain having a peptide according to SEQ ID NO. 47, coupled by a linker domain according SEQ. ID NO. 12, forming a α/β scaffold formation. This engineered antimicrobial peptide may be a therapeutic agent for plants, and more specifically citrus plants infected with and/or at risk of being infected by Xylella fastidiosa (Xf). In this embodiment, such engineered antimicrobial peptide identified as SEQ ID NO. 31 may exhibit a therapeutic effect against Xylella fastidiosa (Xf), or other gram-negative bacteria through an enhanced bactericidal effect as compared to a single endogenous antimicrobial peptide sub-component (SEQ ID NOs 46-47) in apple plants exhibiting in grape plants exhibiting Pierce's disease.

In one specific example, an antimicrobial peptide, identified as amino acid SEQ ID NO. 54, or a variant or homolog of the same, may include a first α/β peptide domain having a peptide according to SEQ ID NO. 45, and said second α/β peptide domain having a peptide according to SEQ ID NO. 59, coupled by a linker domain according SEQ. ID NO. 57, forming a α/β scaffold formation. This engineered antimicrobial peptide may be a therapeutic agent for plants, and more specifically citrus plants infected with and/or at risk of being infected by Candidatus Liberibacte asiaticus ((Las). In this embodiment, such engineered antimicrobial peptide identified as SEQ ID NO. 54 may exhibit a therapeutic effect against Candidatus Liberibacte asiaticus ((Las), or other gram-negative bacteria through an enhanced bactericidal effect as compared to a single endogenous antimicrobial peptide sub-component (SEQ ID NOs 45 and 59) in apple plants exhibiting Huanglongbing (HLB).

In one specific example, an antimicrobial peptide, identified as amino acid SEQ ID NO. 54, or a variant or homolog of the same, may include a first α/β peptide domain having a peptide according to SEQ ID NO. 45, and said second α/β peptide domain having a peptide according to SEQ ID NO. 59, coupled by a linker domain according SEQ. ID NO. 57, forming a α/β scaffold formation. This engineered antimicrobial peptide may be a therapeutic agent for plants, and more specifically citrus plants infected with and/or at risk of being infected by E. amylovora. In this embodiment, such engineered antimicrobial peptide identified as SEQ ID NO. 54 may exhibit a therapeutic effect against E. amylovora, or other gram-negative bacteria through an enhanced bactericidal effect as compared to a single endogenous antimicrobial peptide sub-component (SEQ ID NOs 45 and 59) in apple plants exhibiting Fire Blight.

In one specific example, an antimicrobial peptide, identified as amino acid SEQ ID NO. 54, or a variant or homolog of the same, may include a first α/β peptide domain having a peptide according to SEQ ID NO. 45, and said second α/β peptide domain having a peptide according to SEQ ID NO. 59, coupled by a linker domain according SEQ. ID NO. 57, forming a α/β scaffold formation. This engineered antimicrobial peptide may be a therapeutic agent for plants, and more specifically citrus plants infected with and/or at risk of being infected by Xylella fastidiosa (Xf). In this embodiment, such engineered antimicrobial peptide identified as SEQ ID NO. 54 may exhibit a therapeutic effect against Xylella fastidiosa (Xf), or other gram-negative bacteria through an enhanced bactericidal effect as compared to a single endogenous antimicrobial peptide sub-component (SEQ ID NOs 45 and 59) in apple plants exhibiting in grape plants exhibiting Pierce's disease.

In another specific example, an antimicrobial peptide of the invention may include a first α/β peptide domain having a peptide according to SEQ ID NO. 1-6, and/or 57 or a variant or homolog thereof, and said second α/β peptide domain having a peptide according to SEQ ID NO. 1-6, and/or 57 or a variant or homolog thereof, coupled by a linker domain according SEQ. ID NO. 7-16, and/or 58 forming a α/β scaffold formation. This engineered antimicrobial peptide may be a therapeutic agent for plants, and more specifically citrus plants infected with and/or at risk of being infected by Xylella fastidiosa (Xf). In this embodiment, such engineered antimicrobial peptides of the invention may exhibit a therapeutic effect against Xylella fastidiosa (Xf), or other gram-negative bacteria through an enhanced bactericidal effect as compared to a single endogenous antimicrobial peptide sub-component in grape plants exhibiting Pierce's disease.

The term “applying,” “application,” “administering,” “administration,” and all their cognates, as used herein, refers to any method for contacting the plant with the antimicrobial peptide compositions, and preferably topically contacting the plant with the antimicrobial peptide compositions discussed herein. Administration generally is achieved by application of the compositions in a vehicle compatible with the plant to be treated (i.e., a botanically compatible vehicle or carrier), such as an aqueous vehicle, to the plant or to the soil surrounding the plant or by injection into the plant. Any application can be used, however one application methods include trunk injection and foliar spraying as described herein. Other methods include application to the soil surrounding the plant, by injection, soaking or spraying, so that the applied compounds can come into contact with the plant roots and can be taken up by the roots. Additional topical applications may also be contemplated. The compositions disclosed herein can be formulated for seed or plant treatments in any of the following modes: dry powder, water slurriable powder, liquid solution, flowable concentrate or emulsion, emulsion, microcapsules, gel, or water dispersible granules.

The antimicrobial peptide compositions described herein can also be chosen from a number of formulation types, including isolated antimicrobial peptides, which may further be complex with dustable powders (DP), soluble powders (SP), water soluble granules (SG), water dispersible granules (WG), wettable powders (WP), granules (GR) (slow or fast release), soluble concentrates (SL), oil miscible liquids (OL), ultra-low volume liquids (UL), emulsifiable concentrates (EC), dispersible concentrates (DC), emulsions (both oil in water (EW) and water in oil (EO)), micro-emulsions (ME), suspension concentrates (SC), oil-based suspension concentrate (OD), aerosols, fogging/smoke formulations, capsule suspensions (CS) and seed/plant treatment formulations.

In another embodiment, delivery of the antimicrobial peptide composition to plants can be via different routes. The compositions can be suitably administered as an aerosol, for example by spraying onto leaves or other plant material. The particles can also be administered by injection, for example directly into a plant, such as into the stem. In certain embodiments the compositions are administered to the roots. This can be achieved by spraying or watering plant roots with compositions. In other embodiments, the particles are introduced into the xylem or phloem, for example by injection or being included in a water supply feeding the xylem or phloem. Application to the stems or leaves of the plant can be performed by spraying or other direct application to the desired area of the plant; however any method known in the art can be used. A solution or vehicle containing the antimicrobial peptides at a dosage of active ingredient can be applied with a sprayer to the stems or leaves until runoff to ensure complete coverage, and repeat three or four times in a growing season. The concentrations, volumes and repeat treatments may change depending on the plant.

Additional embodiments of the invention include a polynucleotide comprising a nucleic acid sequence that may encode one or more of the antimicrobial peptides described herein. In one specific example, the invention may include a polynucleotide comprising a nucleic acid sequence identified as SEQ ID NOs. 21-45, and 54, or a variant thereof. Such sequences may further be operably linked to a promotor to generate an expression vectors and further introduced to a plant, preferably a citrus plant, or plant infected with, or at risk of being infected with HLB, Fire blight or Pierce's disease. In this embodiment, such transformed plant or plant cell may produce the antimicrobial peptide that may contact a pathogen, such as a gram negative pathogen such as Erwinia amylovora, Candidatus Liberibacte asiaticus (CLas), and Xylella fastidiosa (Xf). Such a transformed plant, which in a preferred embodiment may include a citrus plant, may exhibit enhanced resistance to Clas, a causative agent of HLB disease, Xylella fastidiosa (Xf) the causative agent of Pierce's disease, or Erwinia amylovora the causative agent of Fire blight. In additional embodiments, a transformed plant may exhibit decreased bacterial loads of Clas, Xf or E. amylovora and/or decreased symptoms or progression of HLB, Fire Blight or Pierce's disease, respectively. Methods, systems and techniques of stable and transient plant transformation, such as Agrobacterium tumefaciens-mediated transformation, are known in the art and included within the scope of the inventive technology.

The inventive concepts that can be embodied in a wide variety of specific contexts. The specific embodiments discussed herein are merely illustrative of specific ways to make and use the invention and do not delimit the scope of the invention.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens. As noted above, the terms protein and peptide also include protein fragments, epitopes, catalytic sites, signaling sites, localization sites and the like. A peptide or protein may further be a fusion or chimera peptide, which a used herein means a peptide having at least a first and second domain or moiety. As described herein, in certain embodiment various peptides, including chimeric peptides or oligonucleotides, such as RNA molecules may be co-expressed. In some embodiments the elements may be co-expressed from a single expression vector having one or more expression cassettes, or from separate expression vectors having one or more expression cassettes. Such expression may also be the result of transient or stable transformation of a cell.

The term, “antimicrobial peptide,” as used herein refers to any peptide that has microbiocidal and/or microbiostatic activity, and preferably microbiocidal and/or microbiostatic activity toward gram-negative bacteria. As used herein, the term “Gram-negative” means bacteria that lose the crystal violet stain (and take the color of the red counterstain) in Gram's method of staining. This is characteristic of bacteria that have a cell wall composed of a thin layer of a particular substance (called peptidoglycan).

As used herein, a compound is referred to as “isolated” when it has been separated from at least one component with which it is naturally associated. For example, a metabolite can be considered isolated if it is separated from contaminants including polypeptides, polynucleotides and other metabolites. Isolated molecules can be either prepared synthetically or purified from their natural environment. Standard quantification methodologies known in the art can be employed to obtain and isolate the molecules of the invention.

The term “expression,” as used herein, or “expression” of a coding sequence (for example, a gene or a transgene) refers to the process by which the coded information of a nucleic acid transcriptional unit (including, e.g., genomic DNA or cDNA) is converted into an operational, non-operational, or structural part of a cell, often including the synthesis of a protein. Gene expression can be influenced by external signals; for example, exposure of a cell, tissue, or organism to an agent that increases or decreases gene expression. Expression of a gene can also be regulated anywhere in the pathway from DNA to RNA to protein. Regulation of gene expression occurs, for example, through controls acting on transcription, translation, RNA transport and processing, degradation of intermediary molecules such as mRNA, or through activation, inactivation, compartmentalization, or degradation of specific protein molecules after they have been made, or by combinations thereof. Gene expression can be measured at the RNA level or the protein level by any method known in the art, including, without limitation, Northern blot, RT-PCR, Western blot, or in vitro, in situ, or in vivo protein activity assay(s).

The term “nucleic acid” or “nucleic acid molecules” include single- and double-stranded forms of DNA; single-stranded forms of RNA; and double-stranded forms of RNA (dsRNA). The term “nucleotide sequence” or “nucleic acid sequence” refers to both the sense and antisense strands of a nucleic acid as either individual single strands or in the duplex.

The term “gene” or “sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons).

A nucleic acid molecule may include either or both naturally occurring and modified nucleotides linked together by naturally occurring and/or non-naturally occurring nucleotide linkages. Nucleic acid molecules may be modified chemically or biochemically, or may contain non-natural or derivatized nucleotide bases, as will be readily appreciated by those of skill in the art. Such modifications include, for example, labels, methylation, substitution of one or more of the naturally occurring nucleotides with an analog, internucleotide modifications (e.g., uncharged linkages: for example, methyl phosphonates, phosphotriesters, phosphoramidates, carbamates, etc.; charged linkages: for example, phosphorothioates, phosphorodithioates, etc.; pendent moieties: for example, peptides; intercalators: for example, acridine, psoralen, etc.; chelators; alkylators; and modified linkages: for example, alpha anomeric nucleic acids, etc.). The term “nucleic acid molecule” also includes any topological conformation, including single-stranded, double-stranded, partially duplexed, triplexed, hairpinned, circular, and padlocked conformations.

The term “sequence identity” or “identity,” as used herein in the context of two nucleic acid or polypeptide sequences, refers to the residues in the two sequences that are the same when aligned for maximum correspondence over a specified comparison window. As used herein, the term “percentage of sequence identity” may refer to the value determined by comparing two optimally aligned sequences (e.g., nucleic acid sequences) over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa.

Notably, disclosure of a nucleotide sequence also specifically includes with the disclosure its corresponding RNA and amino acid sequence, and vice versa.

Polynucleotide sequences may have substantial identity, substantial homology, or substantial complementarity to the selected region of the target gene. As used herein “substantial identity” and “substantial homology” indicate sequences that have sequence identity or homology to each other. Generally, sequences that are substantially identical or substantially homologous will have about 75%, 80%, 85%, 90%, 91%, 92%, 93%, 94%, 95%, 96%, 97%, 98%, 99%, or 100% sequence identity wherein the percent sequence identity is based on the entire sequence and is determined by GAP alignment ixsing default parameters (GCG, GAP version 10, Accelrys, San Diego, CA). GAP uses the algorithm of Needleman and Wunsch ((1970) J Mol Biol 48:443-453) to find the alignment of two complete sequences that maximizes the number of matches and minimizes the number of sequence gaps. Sequences which have 100% identity are identical. “Substantial complementarity” refers to sequences that are complementary to each other, and are able to base pair with each other. In describing complementary sequences, if all the nucleotides in the first sequence will base pair to the second sequence, these sequences are fully complementary.

Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10. The National Center for Biotechnology Information (NCBI) Basic Local Alignment Search Tool (BLAST™; Altschul et al. (1990)) is available from several sources, including the National Center for Biotechnology Information (Bethesda, Md.), and on the internet, for use in connection with several sequence analysis programs. A description of how to determine sequence identity using this program is available on the internet under the “help” section for BLAST™ For comparisons of nucleic acid sequences, the “Blast 2 sequences” function of the BLAST™ (Blastn) program may be employed using the default BLOSUM62 matrix set to default parameters. Nucleic acid sequences with even greater similarity to the reference sequences will show increasing percentage identity when assessed by this method.

As used herein, the term “homologous” with regard to a contiguous nucleic acid sequence, refers to contiguous nucleotide sequences that hybridize under appropriate conditions to the reference nucleic acid sequence. For example, homologous sequences may have from about 70%-100, or more generally 80% to 100% sequence identity, such as about 81%; about 82%; about 83%; about 84%; about 85%; about 86%; about 87%; about 88%; about 89%; about 90%; about 91%; about 92%; about 93%; about 94% about 95%; about 96%; about 97%; about 98%; about 98.5%; about 99%; about 99.5%; and about 100%. The property of substantial homology is closely related to specific hybridization. For example, a nucleic acid molecule is specifically hybridizable when there is a sufficient degree of complementarity to avoid non-specific binding of the nucleic acid to non-target sequences under conditions where specific binding is desired, for example, under stringent hybridization conditions.

Homologs, variants and alleles of the target molecules or proteins of the invention can be identified by conventional techniques. As used herein, a homolog or variant to a polypeptide is a polypeptide from a plant source that has a high degree of structural similarity to the identified polypeptide.

The term, “operably linked,” when used in reference to a regulatory sequence and a coding sequence, means that the regulatory sequence affects the expression of the linked coding sequence. “Regulatory sequences,” or “control elements,” refer to nucleotide sequences that influence the timing and level/amount of transcription, RNA processing or stability, or translation of the associated coding sequence. Regulatory sequences may include promoters; translation leader sequences; introns; enhancers; stem-loop structures; repressor binding sequences; termination sequences; polyadenylation recognition sequences; etc. Particular regulatory sequences may be located upstream and/or downstream of a coding sequence operably linked thereto. Also, particular regulatory sequences operably linked to a coding sequence may be located on the associated complementary strand of a double-stranded nucleic acid molecule.

As used herein, the term “promoter” refers to a region of DNA that may be upstream from the start of transcription, and that may be involved in recognition and binding of RNA polymerase and other proteins to initiate transcription. A promoter may be operably linked to a coding sequence for expression in a cell, or a promoter may be operably linked to a nucleotide sequence encoding a signal sequence which may be operably linked to a coding sequence for expression in a cell. A “plant promoter” may be a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred.” Promoters that initiate transcription only in certain tissues are referred to as “tissue-specific.” A “cell type-specific” promoter primarily drives expression in certain cell types in one or more organs, for example, vascular cells in roots or leaves. An “inducible” promoter may be a promoter that may be under environmental control. Examples of environmental conditions that may initiate transcription by inducible promoters include anaerobic conditions and the presence of light. Tissue-specific, tissue-preferred, cell type specific, and inducible promoters constitute the class of “non-constitutive” promoters. A “constitutive” promoter is a promoter that may be active under most environmental conditions or in most cell or tissue types.

As used herein, the term “transformation” or “genetically modified” refers to the transfer of one or more nucleic acid molecule(s) into a cell. A microorganism is “transformed” or “genetically modified” by a nucleic acid molecule transduced into the bacteria when the nucleic acid molecule becomes stably replicated by the bacteria. As used herein, the term “transformation” or “genetically modified” encompasses all techniques by which a nucleic acid molecule can be introduced into such a bacteria.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are several types of vectors including virus, plasmid, bacteriophages, cosmids, and bacteria.

An “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.” In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). A polynucleotide sequence is operably linked to an expression control sequence(s) (e.g., a promoter and, optionally, an enhancer) when the expression control sequence controls and regulates the transcription and/or translation of that polynucleotide sequence.

Unless otherwise indicated, a particular nucleic acid sequence also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. As provided below, the table contains information about which nucleic acid codons encode which amino acids.

Amino acid Nucleic acid codons

| | Nucleic Acid |
|---|---|
| Amino Acid | Codons |
| Ala/A | GCT, GCC, GCA, |
| | GCG |
| Arg/R | CGT, CGC, CGA, |
| | CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, |
| | GGG |
| His/H | CAT, CAC |
| Ile/I | ATT, ATC, ATA |
| Leu/L | TTA, TTG, CTT, |
| | CTC, CTA, CTG |
| Lys/K | AAA, AAG |
| Met/M | ATG |
| Phe/F | TTT, TTC |
| Pro/P | CCT, CCC, CCA, |
| | CCG |
| Ser/S | TCT, TCC, TCA, |
| | TCG, AGT, AGC |
| Thr/T | ACT, ACC, ACA, |
| | ACG |
| Trp/W | TGG |
| Tyr/Y | TAT, TAC |
| Val/V | GTT, GTC, GTA, |
| | GTG |

In addition to the degenerate nature of the nucleotide codons which encode amino acids, alterations in a polynucleotide that result in the production of a chemically equivalent amino acid at a given site, but do not affect the functional properties of the encoded polypeptide, are well known in the art. “Conservative amino acid substitutions” are those substitutions that are predicted to interfere least with the properties of the reference polypeptide. In other words, conservative amino acid substitutions substantially conserve the structure and the function of the reference protein. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine or histidine, can also be expected to produce a functionally equivalent protein or polypeptide. As provided below, the table provides a list of exemplary conservative amino acid substitutions. Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

Amino Acids and Conservative Substitutes

| Amino Acid | Conservative Substitute |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, |
| | Glu |
| Ile | Leu, Val |
| Leu | Ile, Val |
| Lys | Arg, Gln, Glu |
| Met | Ile, Leu |
| Phe | His, Leu, Met, |
| | Trp, Tyr |
| Ser | Cys, Thr |
| Thr | Ser, Val |
| Trp | Phe, Tyr |
| Tyr | His, Phe, Trp |
| Val | Ile, Leu, Thr |

Oligonucleotides and polynucleotides that are not commercially available can be chemically synthesized e.g., according to the solid phase phosphoramidite triester method first described by Beaucage and Caruthers, Tetrahedron Letts. 22:1859-1862 (1981), or using an automated synthesizer, as described in Van Devanter et al., Nucleic Acids Res. 12:6159-6168 (1984). Other methods for synthesizing oligonucleotides and polynucleotides are known in the art. Purification of oligonucleotides is by either native acrylamide gel electrophoresis or by anion-exchange HPLC as described in Pearson & Reanier, J. Chrom. 255:137-149 (1983). Additional methods are known by those of ordinary skill in the art.

As used herein, the term “endogenous” refers to any material from or produced inside an organism, cell, tissue or system.

As used herein, the term “exogenous” or “heterologous” refers to any material introduced from or produced outside an organism, cell, tissue or system.

The term “recombinant” when used with reference, e.g., to a cell, or nucleic acid, protein, or vector, indicates that the cell, organism, nucleic acid, protein or vector, has been modified by the introduction of a heterologous nucleic acid or protein, or the alteration of a native nucleic acid or protein, or that the cell is derived from a cell so modified. Thus, for example, recombinant cells may express genes that are not found within the native (nonrecombinant or wild-type) form of the cell or express native genes that are otherwise abnormally expressed—over-expressed, under expressed or not expressed at all.

The terms “transgenic,” “transformed,” “transformation,” and “transfection” are similar in meaning to “recombinant.” “Transformation,” “transgenic,” and “transfection” refer to the transfer of a polynucleotide into the genome of a host organism or into a cell. Such a transfer of polynucleotides can result in genetically stable inheritance of the polynucleotides or in the polynucleotides remaining extra-chromosomally (not integrated into the chromosome of the cell). Genetically stable inheritance may potentially require the transgenic organism or cell to be subjected for a period of time to one or more conditions which require the transcription of some or all of the transferred polynucleotide in order for the transgenic organism or cell to live and/or grow. Polynucleotides that are transformed into a cell but are not integrated into the host's chromosome remain as an expression vector within the cell. One may need to grow the cell under certain growth or environmental conditions in order for the expression vector to remain in the cell or the cell's progeny. Further, for expression to occur the organism or cell may need to be kept under certain conditions. Host organisms or cells containing the recombinant polynucleotide can be referred to as “transgenic” or “transformed” organisms or cells or simply as “transformants,” as well as recombinant organisms or cells.

A genetically altered organism is any organism with any change to its genetic material, whether in the nucleus or cytoplasm (organelle). As such, a genetically altered organism can be a recombinant or transformed organism. A genetically altered organism can also be an organism that was subjected to one or more mutagens or the progeny of an organism that was subjected to one or more mutagens and has changes in its DNA caused by the one or more mutagens, as compared to the wild-type organism (i.e., organism not subjected to the mutagens). Also, an organism that has been bred to incorporate a mutation into its genetic material is a genetically altered organism. For the purposes of this invention, the organism is a plant.

The term “plant” includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The class of plants that can be used in the method of the invention is generally as broad as the class of higher and lower plants amenable to the molecular biology and plant breeding techniques described herein, specifically angiosperms (monocotyledonous (monocots) and dicotyledonous (dicots) plants including eudicots. It includes plants of a variety of ploidy levels, including aneuploid, polyploid, diploid, haploid and hemizygous. In one preferred embodiment, the genetically altered plants described herein can be dicot crops, such as citrus.

The terms “approximately” and “about” refer to a quantity, level, value or amount that varies by as much as 30%, or in another embodiment by as much as 20%, and in a third embodiment by as much as 10% to a reference quantity, level, value or amount.

As used herein the term “increased, or decreased with respect to the use or effect of an antimicrobial peptide means increased, or decreased compared to wild-type.

As used herein, the singular form “a,” “an,” and “the” include plural references unless the context clearly dictates otherwise. For example, the term “a peptide” includes both a single peptide and a plurality of peptides.

As noted above, the compositions and substances set forth above can be used to modulate the amount of Erwinia amylovora, Candidatus Liberibacte asiaticus ((Las), and Xylella fastidiosa (Xf) infestation in plants, their seeds, roots, fruits, foliage, stems, tubers, and in particular, inhibit and/or prevent Erwinia amylovora, Candidatus Liberibacte asiaticus ((Las), and Xylella fastidiosa (Xf). infection, in particular, decrease the rate and/or degree of spread of Erwinia amylovora, Candidatus Liberibacte asiaticus (CLas), and Xylella fastidiosa (Xf) infection in plants. While a preferred embodiment may include citrus plants generally, additional plants, include but are not limited to fruits (e.g., strawberry, blueberry, blackberry, peach and other stone fruits), vegetable (e.g., tomato, squash, pepper, eggplant, potatoes, carrots), or grain crops (e.g., soy, wheat, rice, corn, sorghum), trees, flowers, ornamental plants, shrubs (e.g., cotton, roses), bulb plants (e.g., onion, garlic) or vines (e.g., grape vine), turf, tubers (e.g. potato, carrots, beets). Alternatively, the inventive compositions can be used to modulate the amount of Erwinia amylovora, Candidatus Liberibacte asiaticus ((Las), and Xylella fastidiosa (Xf) infection in plants and in particular, prevent or inhibit Erwinia amylovora, Candidatus Liberibacte asiaticus ((Las), and Xylella fastidiosa (Xf) infection and/or decrease the rate and/or degree of spread of disease infection in said plants.

Persons of skill are aware of various methods to apply microbial-based compositions, to plants for surface application or for uptake, and any of these methods are contemplated for use in this disclosure. Methods of administration to plants include, by way of non-limiting example, application to any part of the plant, by inclusion in irrigation water, by injection into the plant or into the soil surrounding the plant, by exposure of the root system to aqueous solutions containing the compounds, by use in hydroponic or aeroponic systems, by culture of individual or groups of plant cells in media containing the inducer, by seed treatment, by exposure of cuttings of citrus plants used for grafting to aqueous solutions containing the compounds, by application to the roots, stems or leaves, or by application to the plant interior, or any part of the plant to be treated. Any means known to those of skill in the art is contemplated. One mode of administration includes those where the compositions are applied at, on or near the roots of the plant, or trunk injection. Application of microbial-based compositions can be performed in a nursery setting, a greenhouse, hydroponics facility, or in the field, or any setting where it is desirable to treat plants to prevent the likelihood of disease, or to treat disease and its effects, for example in plants which have been or can become exposed to HLB, Fire Blight, or Pierce's disease. The methods and compounds of this disclosure can be used to treat infection with any Erwinia amylovora, Candidatus Liberibacte asiaticus ((Las), and Xylella fastidiosa (Xf) species or type and can be used to improve plant defenses in plants which are not infected. Thus, any plant in need, in the context of this disclosure, includes any and all plants for which improvements in health and vigor, growth and productivity or ability to combat disease is desired. Citrus or other plants susceptible to diseases such as HLB, Fire Blight, or Pierce's disease or infection by Erwinia amylovora, Candidatus Liberibacte asiaticus ((Las), and Xylella fastidiosa (Xf) species, whether currently infected or in potential danger of infection.

As defined herein, with respect to any antimicrobial peptide the terms “derived from” or “from” means directly isolated or obtained from a particular source or alternatively having identifying characteristics of a substance or organism isolated or obtained from a particular source. In the event that the “source” is an organism, “derived from” or “from” means that it may be isolated or obtained from the organism itself or from the medium used to culture or grow said organism.

The term “citrus”, as used herein, refers to any plant of the genus Citrus, family Rutaceae, and includes Citrus maxima (Pomelo), Citrus medica (Citron), Citrus micrantha (Papeda), Citrus reticulata (Mandarin orange), Citrus trifolata (trifoliate orange), Citrus japonica (kumquat), Citrus australasica (Australian Finger Lime), Citrus australis (Australian Round lime), Citrus glauca (Australian Desert Lime), Citrus garrawayae (Mount White Lime), Citrus gracilis (Kakadu Lime or Humpty Doo Lime), Citrus inodora (Russel River Lime), Citrus warburgiana (New Guinea Wild Lime), Citrus wintersii (Brown River Finger Lime), Citrus halimii (limau kadangsa, limau kedut kera); Citrus indica (Indian wild orange), Citrus macroptera, and Citrus latipes. Hybrids also are included in this definition, for example Citrus. times. aurantiifolia (Key lime), Citrus, times, aurantium (Bitter orange), Citrus. times. latifolia (Persian lime), Citrus. times. limon (Lemon), Citrus. times. limonia (Rangpur), Citrus. times. paradisi (Grapefruit), Citrus. times. sinensis (Sweet orange), Citrus. times. tangerina (Tangerine), Poncirus trifoliata. times. C. sinensis (Carrizo citrange), and any other known species or hybrid of genus Citrus. Citrus known by their common names include, Imperial lemon, tangelo, orangelo, tangor, kinnow, kiyomi, Minneola tangelo, oroblanco, sweet orange, ugli, Buddha's hand, citron, lemon, orange, bergamot orange, bitter orange, blood orange, calamondin, Clementine, grapefruit, Meyer lemon, Rangpur, tangerine, and yuzu, and these also are included in the definition of citrus or Citrus.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “any combination thereof” as used herein refers to all permutations and combinations of the listed items preceding the term. For example, “A, B, C, or any combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

In one embodiment, the novel methods of the invention include the identification of non-toxic α/β peptide chimeras in clearing bacteria and augmenting innate immunity during infection. First, the present inventors performed molecular modeling to construct a library of peptide chimeras by joining two antibacterial α/β segments from citrus/apple/grape proteins and selected energetically stable ones with high activity. Second, the present inventors synthesized the selected chimeras and measured minimum inhibitory concentration (MIC) against two exemplary gram-negative E. coli strains. Third, the present inventors determined the toxicity of high activity chimeras against plant and human cells. Fourth, non-toxic and high activity chimeras were tested in a detached leaf assay for anti-CLas activity. Finally, the present inventors determined the expression of selected genes to show that innate immunity in citrus/apple is augmented during infection.

### Example 1: Identification of Chimeric Peptides with In Vitro Bactericidal Activities on E. coli

Antibacterial peptides in plant proteins can either be linear or disulfide (S—S) bridged (15-17). As shown in Table I, four types of linear antibacterial peptides, and related homologs, were initially considered (i) amphipathic peptides such as KKLIKKILKIL (SEQ ID NO. 1)/KKLFKKILKYL (SEQ ID NO. 2), designated as unit A; (ii) FWQ containing basic peptides, such as FWQRRIRRWRR (SEQ ID NO. 3)/FQWORNIRKVR (SEQ ID NO. 4), designated as unit C/B; (iii) unit D, R/W-rich peptides, such as RRWWRWWR (SEQ ID NO. 5); and (iv) unit E, IERSTNLDWYKGPTLL (SEQ ID NO. 6), identified from plant extracts. A library of chimeras was constructed by joining two different units with a flexible linker. In one embodiment, a flexible linker of the invention may include a peptide having a sequence selected from the group consisting of: SEQ ID NO.'s 7-16, and/or 58. The initial structure of each chimera was obtained using homology modeling (19) and energy minimized in vacuum using GROMOS96 force field (20). Previously, the present inventors performed molecular dynamics simulation of various peptides in water: (lipid bilayer) system to visualize the three determinants of antibacterial activity of the peptide (14), namely: membrane attachment, insertion, and rupture. MD simulations also revealed that the three determinants vis-à-vis the antibacterial activity depended on the helical content and stability of the chimera, the total charge, and the relative disposition of charged vs. hydrophobic amino acids on the surface or in the interior of the structure.

These parameters in the energy minimized structures of the chimeras also allowed empirical ranking of the chimeras in terms of antibacterial activity. The top ranked chimeric peptides were custom synthesized in milligram quantities, and were measured to determine the minimum inhibitory concentrations (MIC) of the peptides that killed all 5×10⁵ colony forming units (cfu) of a bacterial culture after 24 hours of incubation (21). The MIC assay was performed on a 96-well plate by serial dilution of the peptide in the concentration range 20-0.02 mM and monitoring the growth of the E. coli strain BL21 and (in some cases ATCC25922) by measuring optical density (OD)/cfu. This produced the range of MIC above which no bacterial growth was observed. Table I shows the MIC ranges of the individual units A-E and chimeras constructed by joining two of them. The chimeric peptides with MIC above 20 mM were not further pursued. Tables sIa and sIb show various fragments of the citrus and apple proteins that are homologous to various chimeras shown in Table I, which include: single units on the N/C-terminal of the chimeras, linkers joining them, and the chimera fragments containing the N-terminal, the linker, and the C-terminal as well.

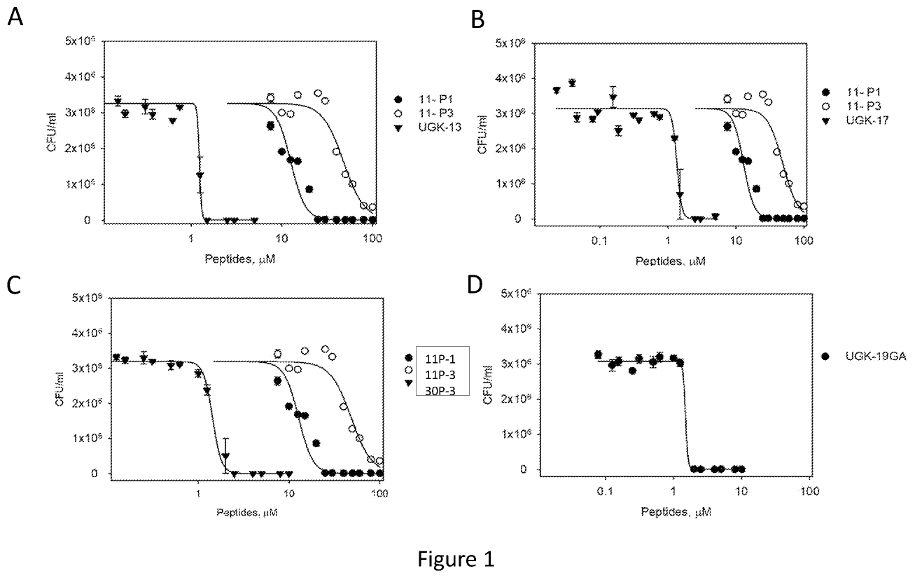

The chimeric peptides with MIC in the range of 0.6-2.5 μM were considered promising as bactericidal agents and selected for the bioluminescence assay for determining the exact MIC values. The bioluminescence assay (22) measures the number of viable microbial cells in culture based on quantitation of the ATP present. Note that ATP is present in live (metabolically active) cells but not in dead cells. Table II lists the bactericidal activities of the promising antibacterial chimeras on E. coli BL21. Table II also shows the activities (IC₅₀ and IC₉₉˜MIC) of the single units A-E and that of the equimolar mixture of A (11P-1) and C (11P-3). Note that chimeras AC and CA were identified as the most promising antibacterial agents. Also, note the synergistic effect of the units A and C in chimeras as evidenced by the much lower MIC of the chimera than that of the equimolar mixtures of units A and C. FIG. 1 shows the cfu from the bioluminescence assay at different concentrations which reveals the slope of the transition from live to dead bacteria (see FIG. 1) upon peptide binding to bacteria and the Hills coefficient, an index of cooperative binding.

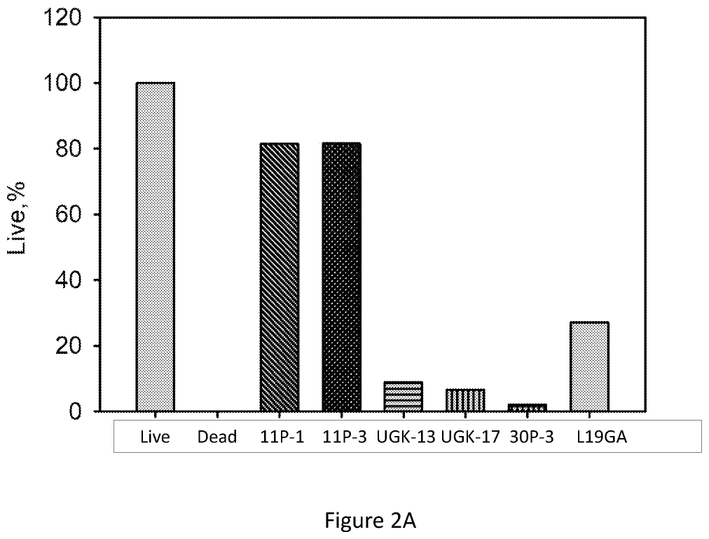

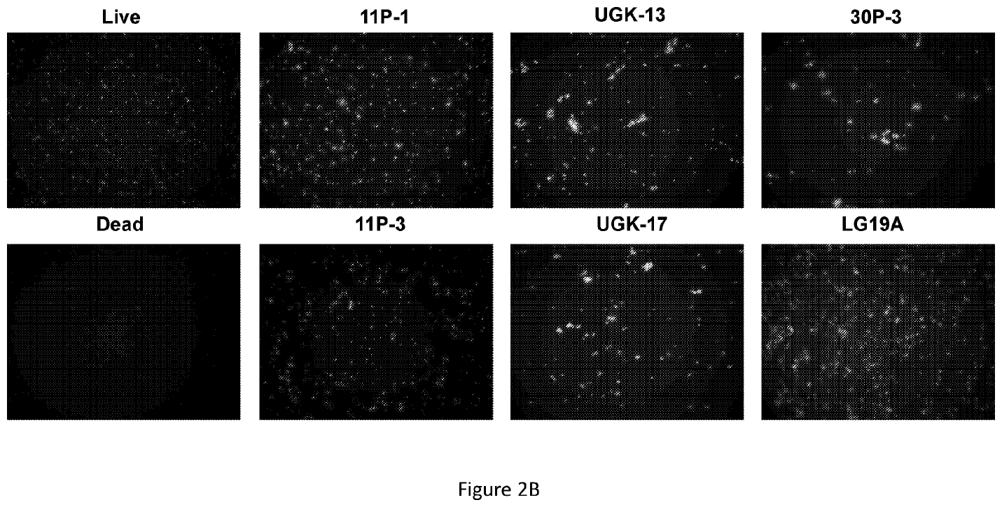

The ratio of live/dead E. coli BL21 cells due to peptide treatment was also monitored by a fluorescent based assay (23). For this, two fluorescent dyes SYTO 9 and propidium iodide (PI), were used. SYTO 9 stains green both live and dead cells whereas PI only intercalates and stains red the DNA of the dead cells or the cells with ruptured membranes. FIG. 2A shows the percentage of live cells after 1 hour of 20 mM peptide treatment on 5×10⁵ cfu of E. coli BL21 relative to the live and dead cell controls whereas FIG. 2B shows the same data presented visually by fluorescent images of green and red labeled BL21 upon peptide treatment. Single units, 11P-1 and 11P-3, and their α/β chimeras, 30P-3, UGK-13, UGK-17, are shown in FIGS. 2A and 2B. In addition, the effect of L19GA on E. coli BL21 is also shown in FIGS. 2A and 2B. Note that, the L19GA peptide contains two b-strands with two intrachain disulfide bridges.

### Example 2: Plant and Human Cell Toxicity of the Peptide Chimeras with In Vitro Bactericidal Activity on E. coli

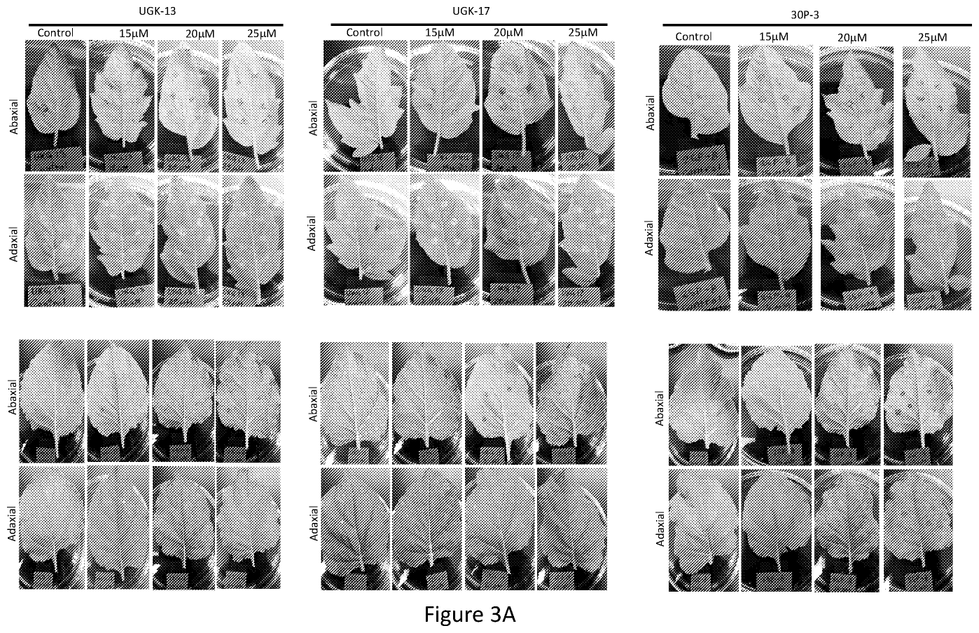

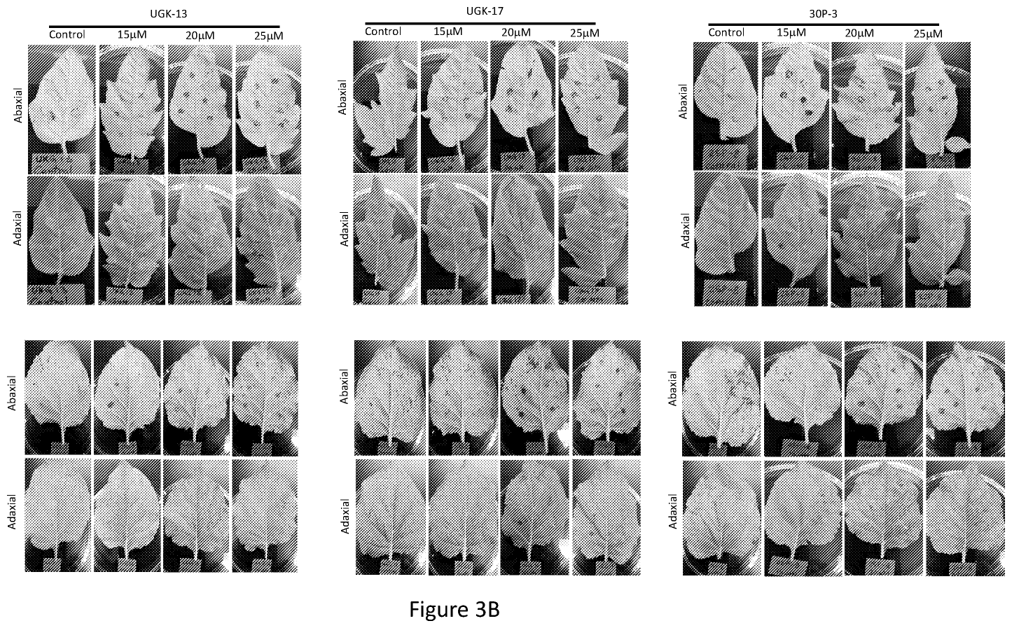

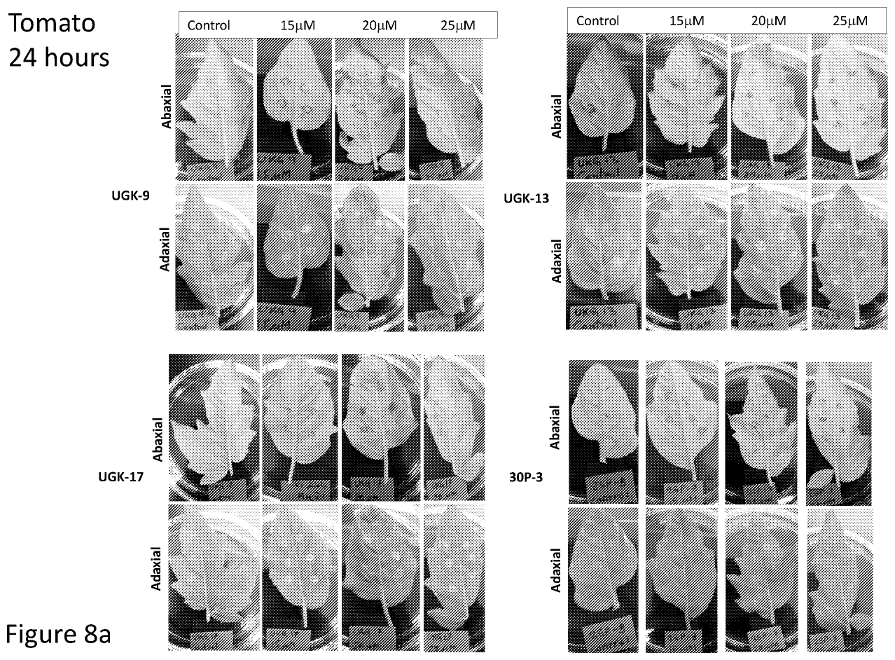

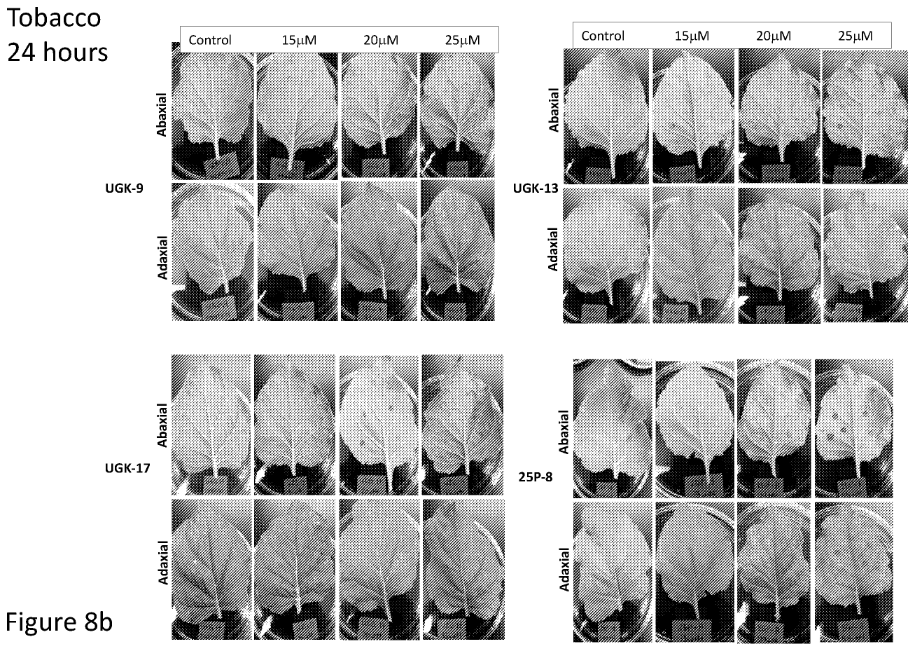

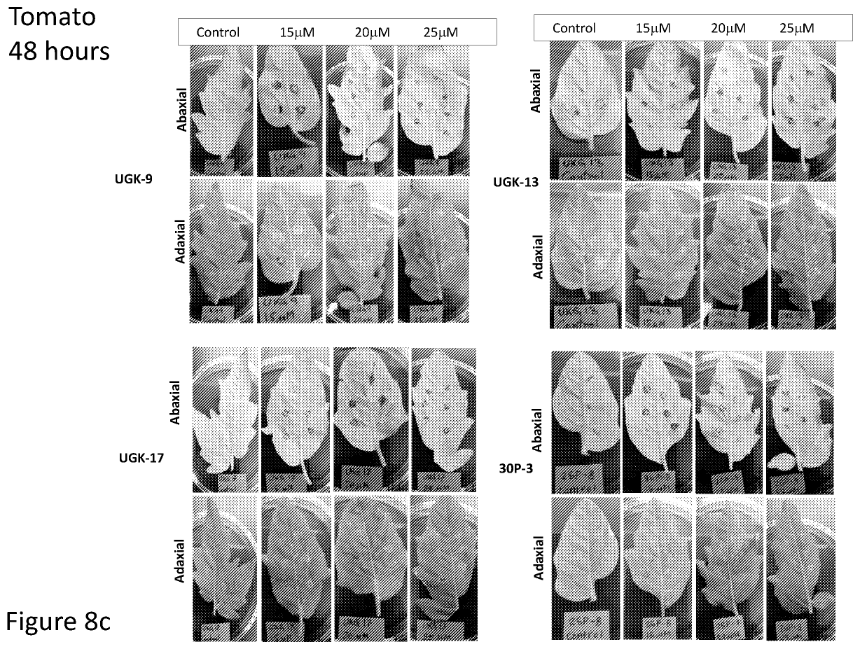

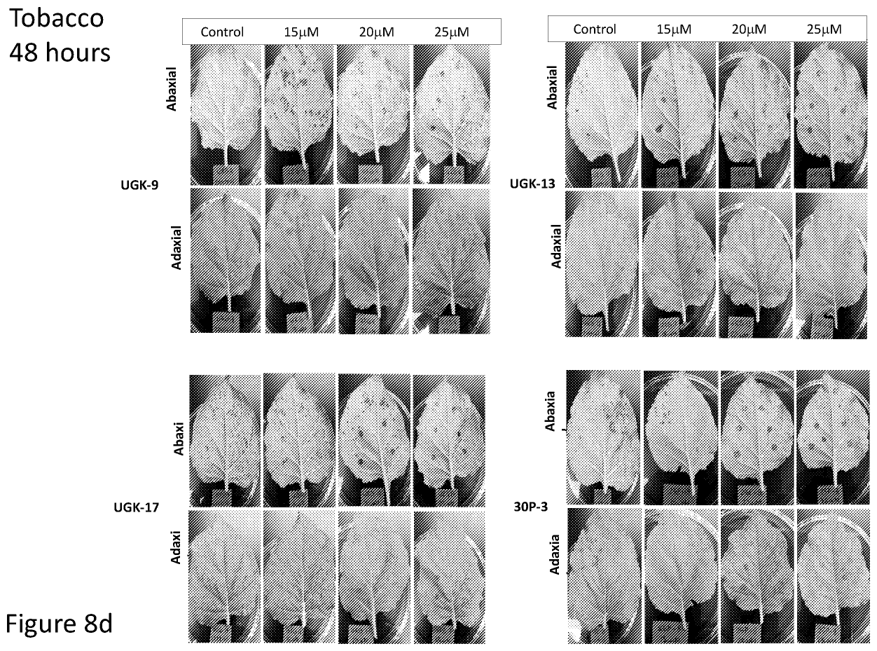

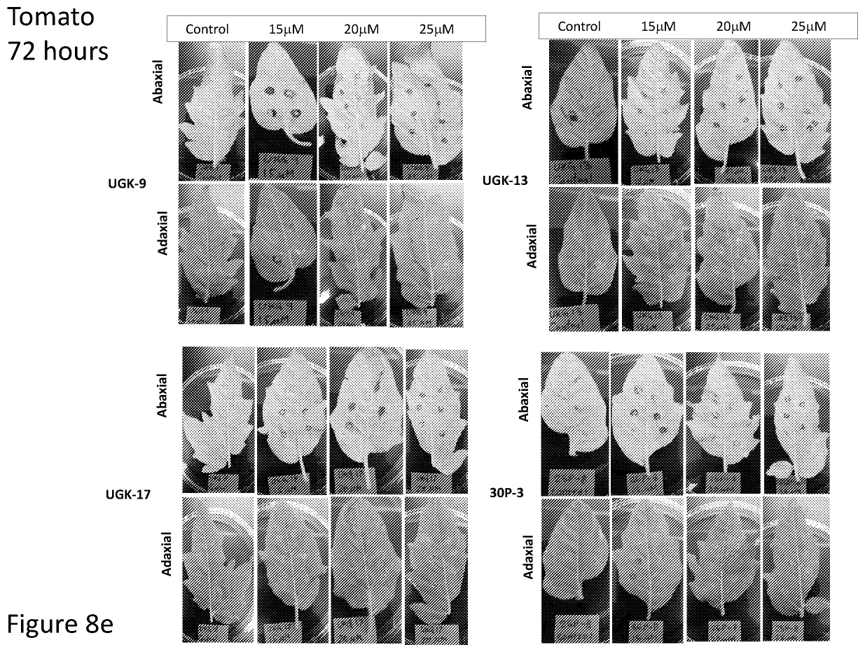

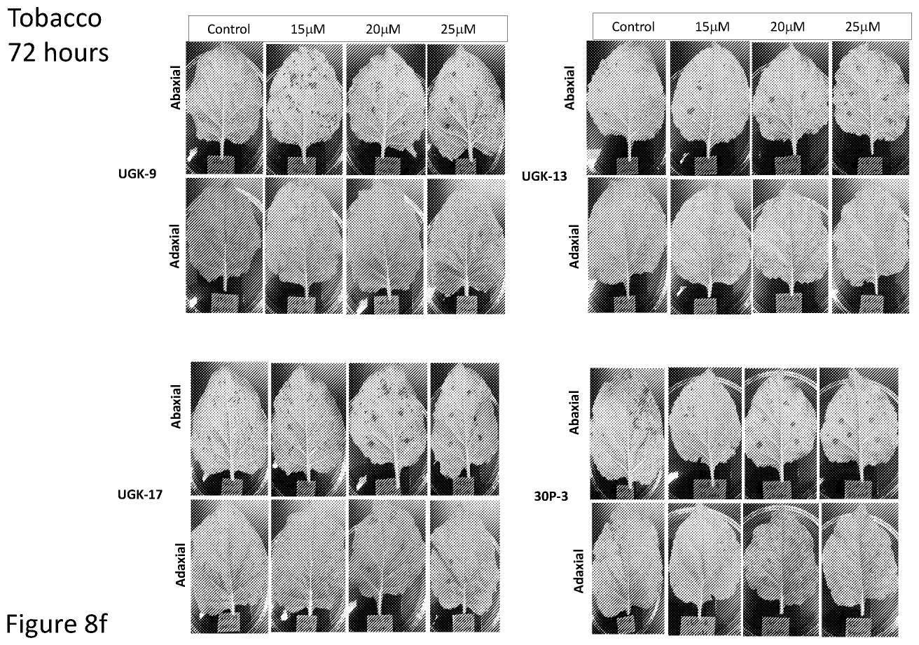

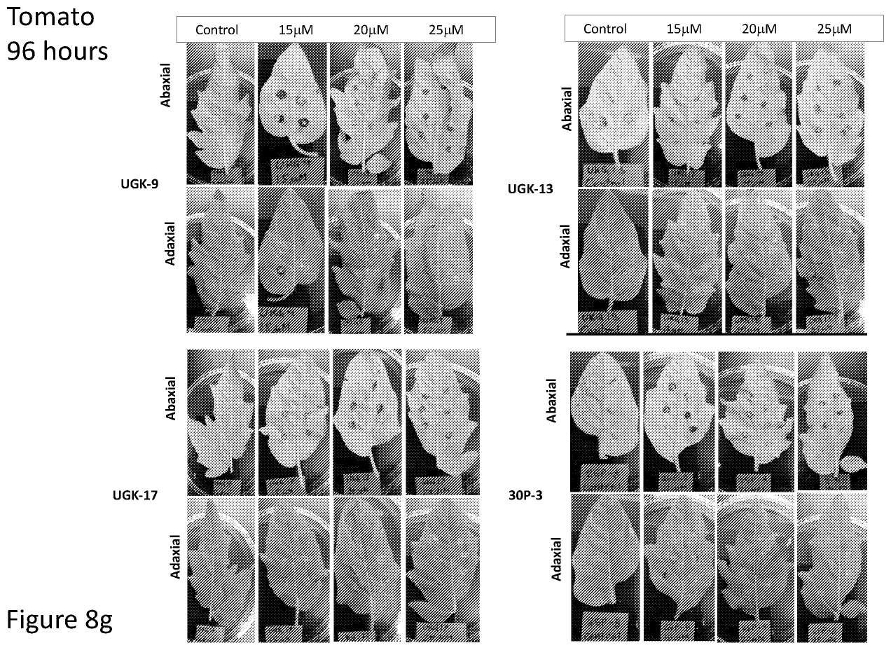

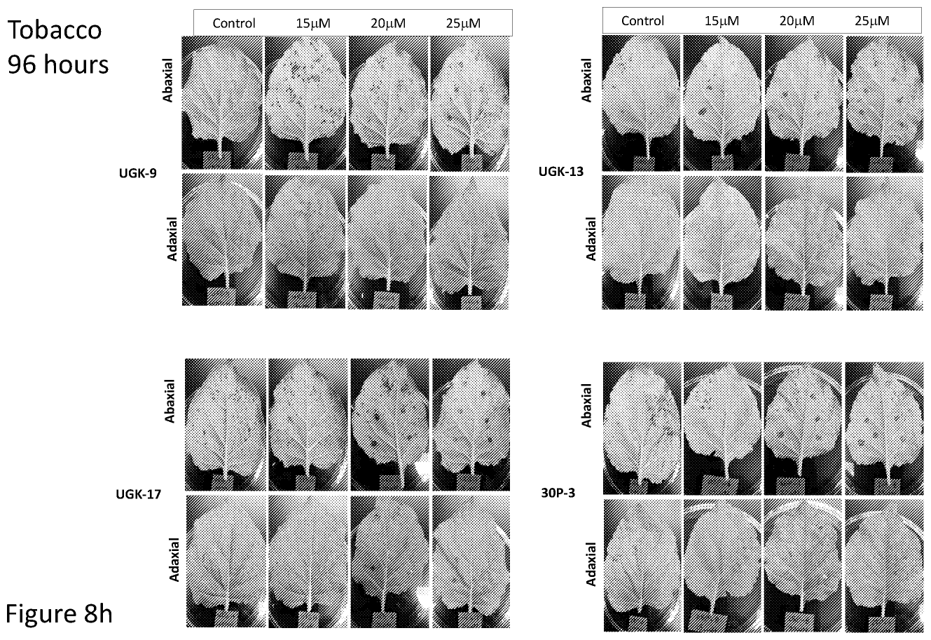

Both plant and human toxicity analyses were performed on the chimeras, UGK-13, UGK-17, UGL-9, and 30P-3 with MIC=0.6-2.0 μM in vitro on E. coli. Plant toxicity was measured by infiltrating 15-25 μM peptide solution to tomato and tobacco leaves 1 ml syringe (24). Each leaf was abaxially and adaxially infiltrated at 4-6 spots. The infiltrated plants were kept for 96 hours in growth chamber. The infiltrated leaves were visually analyzed at 24, 48, 72, and 96 hours. If a peptide is toxic at a given concentration, the corrosive effect should spread in the leave beyond the infiltration spots. Table III summarizes the leaf infiltration data, which show that the chimeras, UGK-13, UGK-17, UGK-9, and 30P-3, are not toxic to tomato and tobacco at concentrations 15-25 μM even after 96 hours of leaf infiltration. FIGS. 3A and 3B show the pictures of the tomato and tobacco leaves after 24 and 96 hours of 15-25 μM peptide infiltration relative to the water infiltrated control using UGK-13, UGK-17, and 30P-3. FIG. 8 in supplementary material contains data on all the four peptides, UGK-13, UGK-17, UGK-9, and 30P-3 for all 24, 48, 72, and 96 hours of infiltration. Note that like the control, the peptide infiltrated tomato and tobacco leaves showed no corrosion beyond the infiltrated spots.

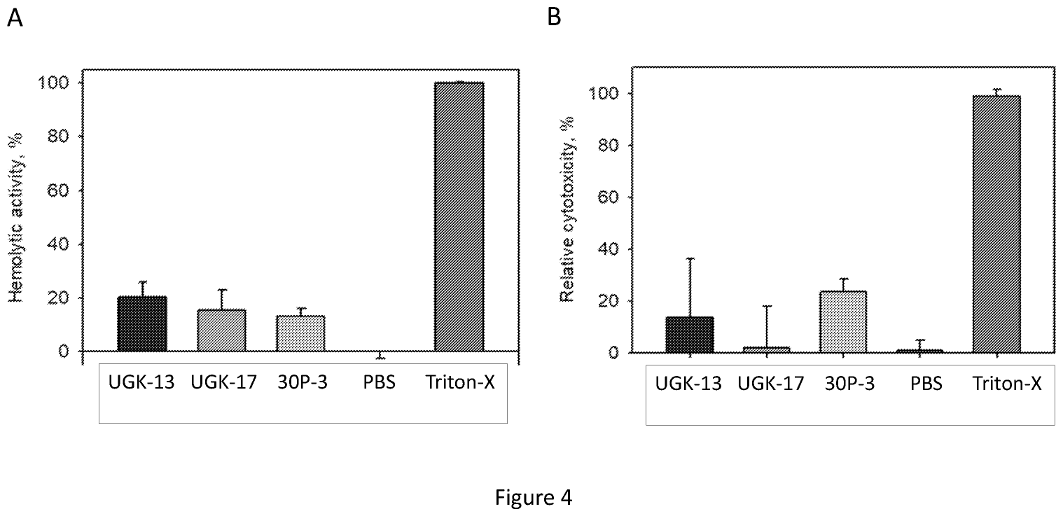

Toxicity of the chimeric peptides of the invention were also measured using human erythrocytes and human kidney embryonic (HEK) cells. Hemolysis of erythrocytes, via hemoglobin release after membrane rupture, provides a rapid measure of toxicity of the chimeric peptides (25). FIG. 4A show the % hemolysis of human erythrocytes by, UGK-13, UGK-17, and 30P-3 at 20 μM relative to PBS (negative) and 0.01% Triton-X-100 (positive control). Cytotoxicity of the HEK cells due to the three chimeric peptide was measured by the MTT assay (26) that assesses the metabolic activity of live cells by monitoring the level of NAD(P)H-dependent cellular oxidoreductase enzymes present in live cells. FIG. 4B shows the % cytotoxicity of the HEK cells treated with 20 μM UGK-13, UGK-17, and 30P-3 relative to PBS and Triton-X. Note that the three chimeras at 20 μM concentration (10 times higher than their MIC) have low (<20%) toxicity to human erythrocytes and HEK.

### Example 3: Structural Models of the Chimeras with Bactericidal Activity and Low Toxicity

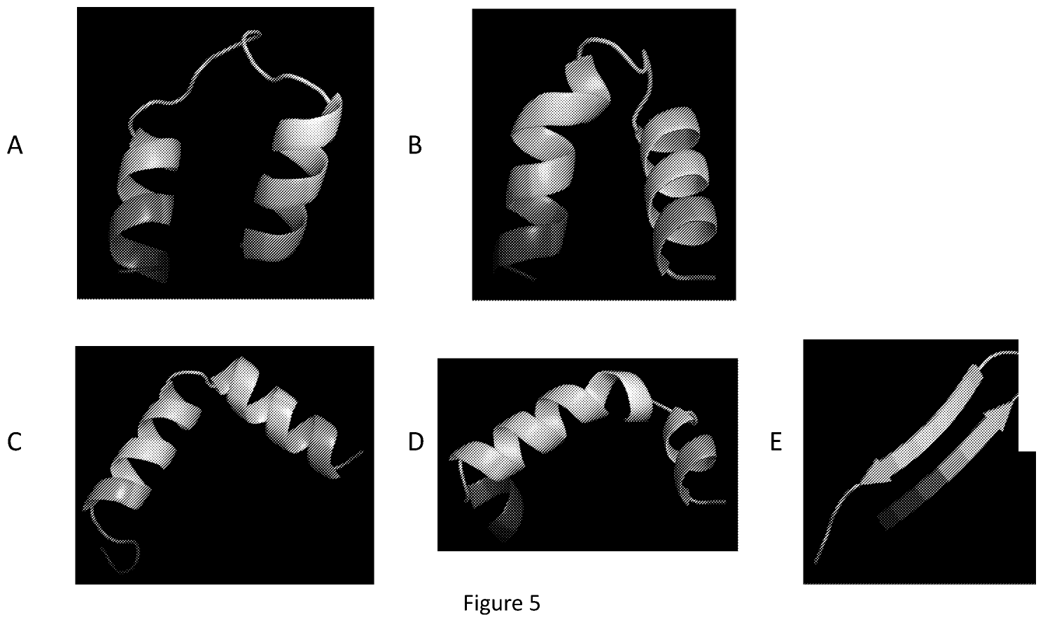

Molecular models of the four chimeras, 30P-3, UGK-13, UGK-17, and UGK-9, are shown in FIG. 5A-D. As stated above, the models are energy minimized after obtaining the initial homology-based structures (19, 20). Note that, 30P-3, UGK-13, and UGK-17 belong to the AC chimera family whereas UGK-9 belongs to the CD chimera family. The three AC chimeras contain α-helical units of A and C. However, the linkers are different in terms of sequence, length, and conformation, as judged by the peptide backbone (ϕ, ψ) angles in the energy minimized models. The linker, GSGYGSPG (SEQ ID NO. 7), in 30P-3 adopts an extended (or β) conformation whereas the linker, GYG, in UGK-13 forms a sharp GY turn followed by a short b strand connecting the C-terminal helix. Finally, the single amino acid S linker UGK-17 forms a b strand (or a kink) between the N- and C-terminal helices. UGK-9, a member of the CD chimera family, forms three a helices. The N-terminal helix is connected by a single amino acid b-stranded “F” to the central helix, which connected to the C-terminal helix by a SPGR turn. L19GA is single peptide fragment with antibacterial activity on E. coli BL21 (see Table I) with homologs in citrus and apple (see Tables sIa and sIb). Two b-strands are joined by a YKQR turn. Thus, in the study we have considered peptides with α-helices and b-strands (designated as a/b peptides). Individual α-helix or b-strand may contribute to the antibacterial activity. In addition, a b-strand may define the conformation of a linker in the chimera.

### Example 4: Ex Planta Antibacterial Activities of Selected Chimeric Peptides Against CLas and E. Amylovora

After determining the antibacterial activity of 30P-3, UGK-13, UGK-17, and UGK-9 and lack of their toxicity to plant leaves and human cells, the present inventors next sought to examine whether these chimeras clear the causative bacteria from the infected citrus and apple leaves. For this, grapefruit leaves from Texas with HLB and Red delicious apples with fire blight from New Mexico were collected for testing. Ex planta bactericidal assays are particularly relevant since CLas cannot be cultured in the laboratory. The ex-planta assay involved collection of infected citrus and apple leaves and measuring bacterial load by qPCR in peptide treated and untreated leaves. Specifically, infected leaves (pre-tested with or without symptoms) were collected from the field. The petioles of the leaves were dipped into sealed Eppendorf tubes containing 1-2 ml of 20-25 μM peptide solution and water (untreated) control. It was estimated that the absorption of 0.8-1 ml of peptide solution was needed for complete clearance. It about a day for the apple leaves whereas it up to 3 days for the citrus leaves to absorb 0.8-1 ml of peptide solution.

The treated and untreated leaves were then crushed, and total DNA/RNA was extracted. The bacterial load was measured from the extracted DNA/RNA by qPCR using primers specific to HLBas locus in CLas (27) and a DNA locus in the plasmid pEA29 in E. amylovora (28). Note that by qPCR of DNA both live and dead bacteria are measured whereas by qPCR of RNA only the live bacteria are counted. Also, primers specific to citrus and apple reference genes were used to ensure that that amplification of these genes were unaltered by treatment to confirm that the peptides were specific to the bacteria. Cytochrome oxidase (COX) of citrus and ubiquitin of apple were chosen as the reference genes for qPCR of DNA and glyceraldehyde-3-phosphate dehydrogenase (GAPDH) gene in citrus and apple was selected as the reference for qPCR of RNA.

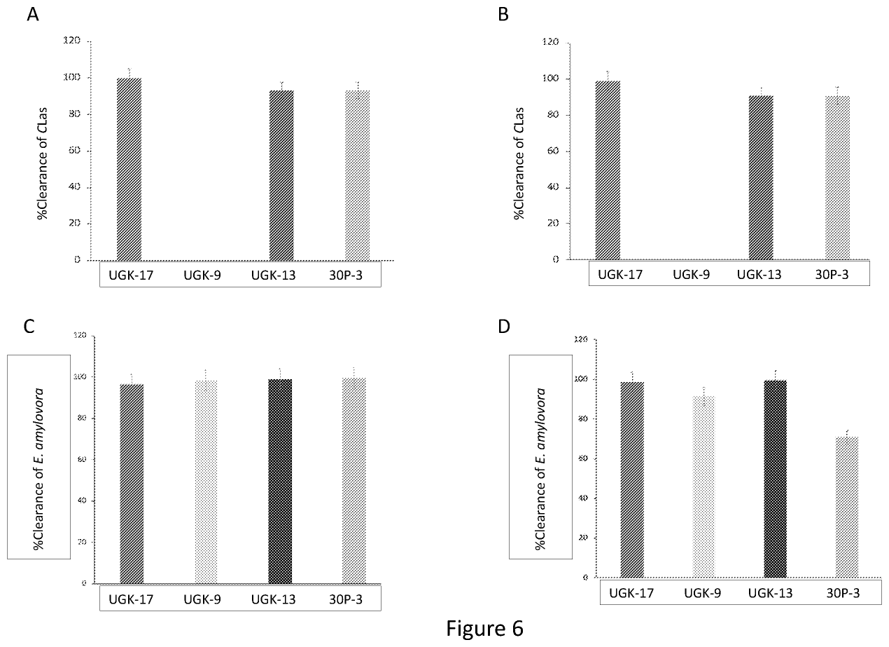

Table IV shows the effect of 25M 30P-3, UGK-9. UGK-13, and UGK-17 on the Ct values and the cfu of CLas and E. amylovora by qPCR of DNA and RNA. Typically, a treatment showing an increase in Ct value over 5 and decrease in cfu to ˜100 is considered an effective one. FIG. 6 shows the % bacterial clearance by four different chimeras relative to the untreated control. Note that UGK-17 is most effective on CLas both by DNA and RNA qPCR. Note that from the DNA qPCR all four chimeras, 30P-3, UGK-9. UGK-13, and UGK-17, appear to be equally effective on E. amylovora. However, the RNA qPCR clearly reveals higher effectiveness of UGK-13 and UGK-17 than that of UGK-9 and 30P-3. qPCR of RNA is relied upon to a greater extent to differentiate the effectiveness of the peptides in bacterial clearance. Tables sIIIa and sIIIb show qPCR data for both 20 and 25 μM peptide concentration for qPCR of DNA. In summary, data presented, so far, indicate that UGK-17 and UGK-13 are effective in clearing E. amylovora whereas UGK-17 is effective in clearing CLas.

### Example 5: Host Derived Chimeric Peptides Augment Innate Immunity

Plant innate immunity upon bacterial infection mainly involves induction and coordination of PTI, PTI, and SA/JA/ET signaling (29). PTI is induced by bacterial MAMP leading to the activation of transcription factors via the MAPK/MAPKK/MAPKKK signalosome cascade and finally the induction of the PR proteins. However, PTI may be inhibited by the bacterial effectors, which, however, may be countered by ETI through the binding of the bacterial effectors to the intracellular NLR resistance protein (30). Effector-NLR binding may rescue PTI signaling by reenforcing MAPK/MAPKK/MAPKKK signalosome complex. The universal WRKY transcription factors play a key role in both activating or suppressing specific defense genes (31). SA/JA/ET signaling is a key component of plant innate immunity against bacterial infection (32-33). The general scheme of this signaling involves the activation of inducible transcription factors and the production of PR proteins. The SA-induced responses operate against biotrophs whereas the JA/ET-induced responses act against necrotrophs. This study focused on examining whether the innate immunity in citrus/apple involving the PTI, ETI, and SA/JA/ET signaling was augmented during infection by the chimeric peptides. For this, RNA was extracted from the treated and untreated citrus/apple samples from the detached leaf assay described above. Then the differential expression of selected genes in the PTI, ETI, and SA/JA/ET signaling were analyzed by qPCR.

FIG. 7A shows the heat-plot of fold changes on a Log₂ scale of the selected genes due to the treatment of 25 μM UGK-17 and UGK-13 on infected grapefruit leaves after 3 days of post-treatment and absorption of 0.8-1 ml of the peptide solution. Note that UGK-17 at 25 μM clears CLas almost 100% whereas UGK-13 at the same concentration clears over 80%. The selected genes belong to: pattern recognition receptors or PRR (FRK1), singling proteins (MAPK6, MAPKK3, COL1, LEA5, EMB564), transcription factors (WRKY4/22/24/29, ERF003/6, Zinc fingers), and PR proteins (PR1/2/3, defensin Ec-AMP-D61, chitinase1, LTP2). The selected list also contains detoxifying enzymes such as CYPP450 82G1, GST1, glycosidases like CsSB1, CsSD1, and lipase GDSL, which may be expressed as a defense response. Note that the phloem specific PP2 protein, a marker for CLas infection, is overexpressed. Most of the selected genes are overexpressed (with fold changes 2-32) by peptide treatment and most notably the PR proteins, the end products of the PTI, ETI, and SA/JA/ET signaling.

FIG. 7B shows the heat-plot of fold changes on a Log₂ scale of selected genes due to the treatment of 25 mM UGK-17, UGK-13, and 30P-3 on infected apple Red Delicious leaves after 48 hours of post-treatment and absorption of 0.8-1 ml of the peptide solution. The selected genes contain plasma membrane and intracellular receptors, genes such as LHC, Jaz17, GA-stimulated genes involved in cytosolic signaling, transcription factors such as JA-induced bHLH, ERF, AP2/ERF and above all the PR genes (ribonuclease, defensin, chitinases, detoxifying enzymes such as peroxidases, SDH). In addition, genes for XTH, hydroxyproline-rich protein involved in membrane structure and biogenesis were included. The selected genes are an important part of the apple reactome (35). The treatment of the three chimeras shows distinctly different patterns in gene expression. A lot more genes are overexpressed by UGK-17 than by UGK-13 although the two have similar bactericidal activities (see FIG. 5C-D). 30P-3 with lower activity by qPCR of RNA also show lower expression of the selected genes.

### Example 6: Identify α/β Peptide(s) with Homologs

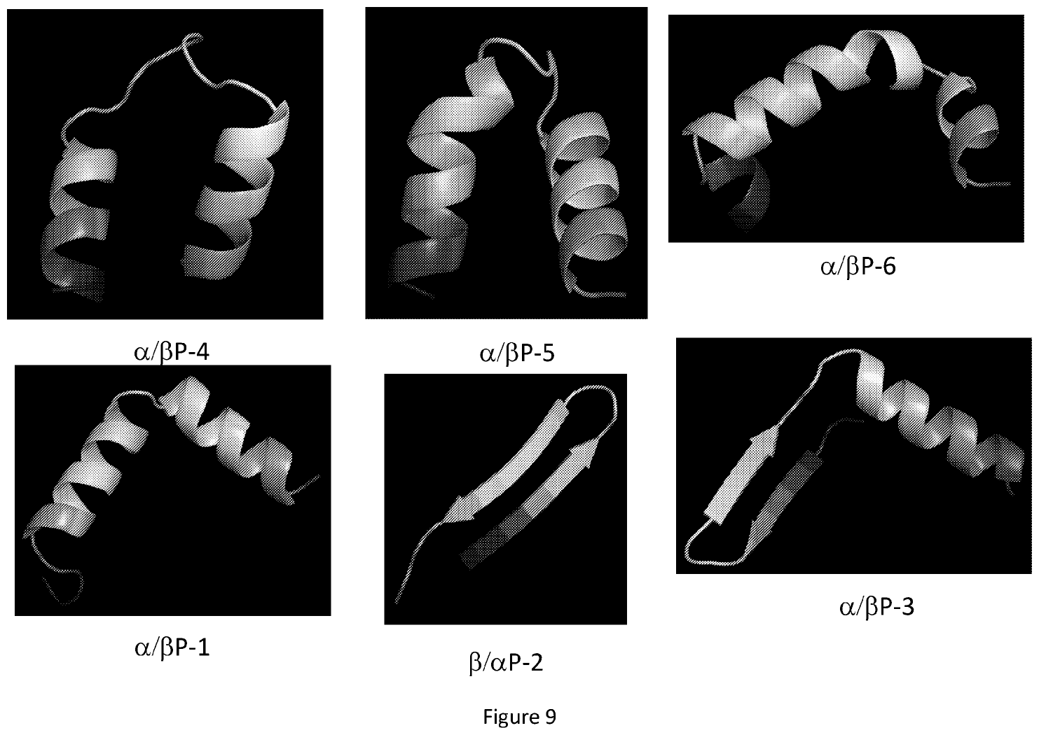

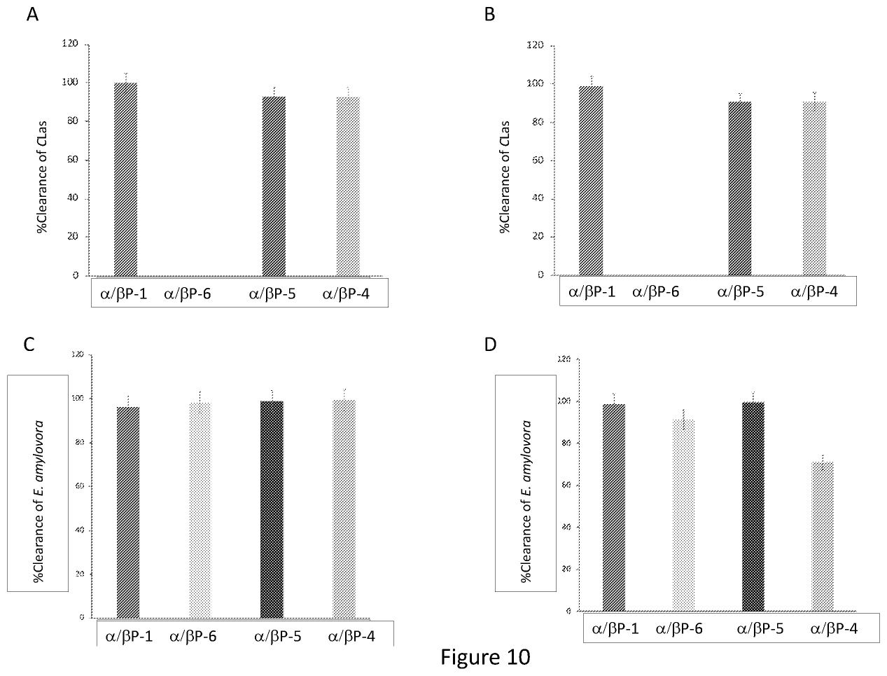

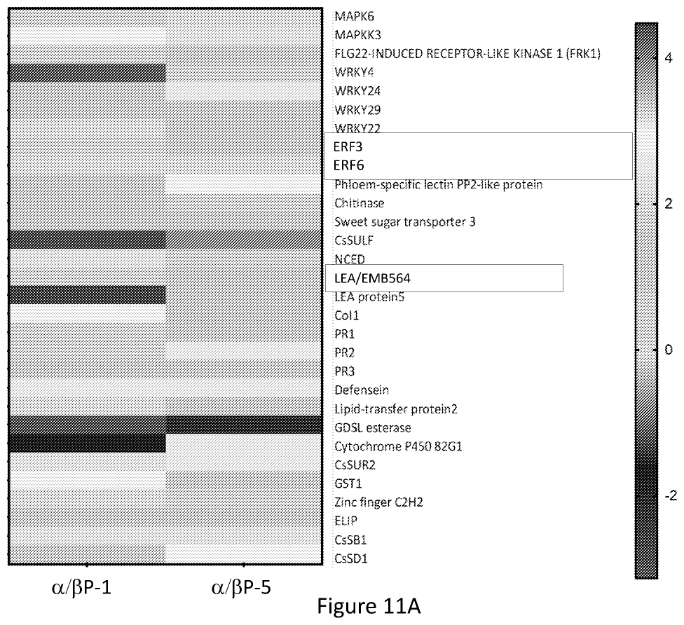

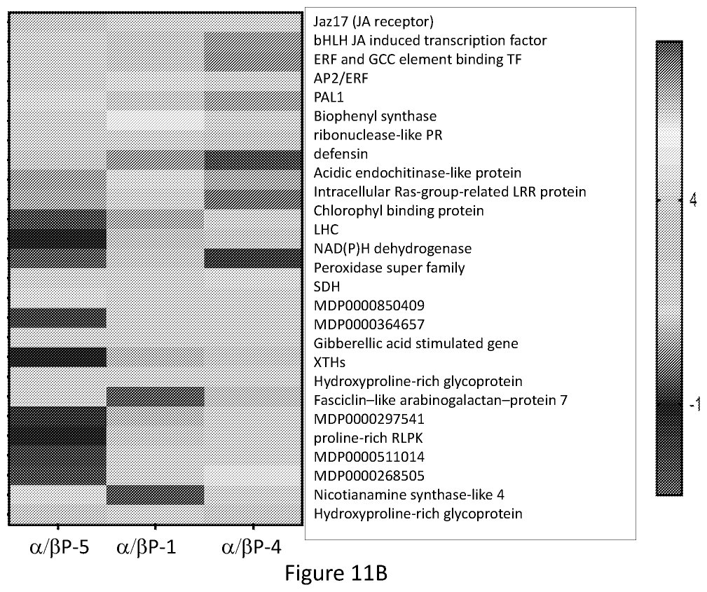

First, plant protein sequences were mined to select α-helical and β-stranded plant peptides with reported membranolytic activity, which, however, are only active at high doses at which they may be toxic to plant and human and most importantly they are susceptible to bacterial resistance. Second, in vitro resistance was developed in a model gram-negative bacterium such as E. coli against a set of a-helical or β-stranded peptides. Alpha helical P11 (KKLIKKILKIL) (SEQ ID NO. 1) was one such peptide against which E. coli resistance was developed in vitro. The following steps were followed to counter bacterial resistance. First, two different a-helical or b-stranded peptides were joined to construct a library of α/β peptides. Second, molecular dynamics simulations were performed to show that the α/β peptides possess higher membrane attachment, insertion, and rupture activity than the individual a or b components. Third, in vitro bactericidal and toxicity assays were conducted to screen non-toxic and highly active α/β peptides for greenhouse and field efficacy studies. It was also confirmed that the screened α/β peptides had sequence homologues in grape, citrus, apple, and tomato. As shown in FIG. 9, cartoon diagrams of the candidates a/bP1-6 are shown. The subset of α/BP-1 and α/BP-3 exhibiting the best bactericidal data are presented in Table I and FIGS. 10-12:

α/βP-1
(SEQ ID NO. 32)
KKLPEKILKILESGYGSPGFWQRRIRRWRR
α/βP-2
(SEQ ID NO. 45)
KC2RRLC6YKQRC11VTYC15RGRQ
(S-S bridges: C2-C15 and C6-C11)
α/βP-3
(SEQ ID NO. 54)
KCRRLCYKQRCVTYCRGROPEKILKILESLK
α/βP-4
(SEQ ID NO. 25)
KKLPKKILKILGSGYGSPGFWQRRIRRWRR
α/βP-5
(SEQ ID NO. 33)
KKLPEKILKILESLKGSPGFWQRRIRRWRR
α/βP-6
(SEQ ID NO. 31)
RLPEAFQWORNIRKVRRPDSPGRRWWRWWR

Both uninfected and infected citrus (grapefruit) leaves were obtained from Texas A&M University-Kingsville Citrus Center, Weslaco, TX and the infected and uninfected apple (red delicious) leaves were obtained from Las Cruces, NM (curtsey: Burke). Leaf samples were stored at −80° C. in sealed in Ziplock bags. Before the experiment the Ziplock bags were put in box and transferred a −20 C refrigerator. Before treatment, the leaves were thawed and dipped in the 1-2 ml peptide solution at the specified concentration at the room temperature for 48-96 hours in the biosafety cabinet. The leaves remained dipped till 0.8-1 ml peptide solution was absorbed. The leaves were then crushed inside the biosafety cabinet in liquid nitrogen using mortar and pestle. The crushed leaves were split into two halves: one for DNA extraction and the other for RNA as per instructions in (E.Z.N.A.® Plant DNA DS Kit; RNeasy plant mini kit). The extracted DNA and RNA were analyzed by qPCR in the BSL-1 lab. Forward and reverse primers for CLas detection were: GTCGAGCGCGTATGCAATACG (SEQ ID NO. 17) and CTACCTTTTTCTACGGGATAACGC (SEQ ID NO. 18), which are chosen to amplify 16s DNA/RNA. Forward and reverse primers for E. amylovora detection were: CACTGATGGTGCCGTTG (SEQ ID NO. 19) and CGCCAGGATAGTCGCATA (SEQ ID NO. 20), which were chosen to amplify the locus in the plasmid pEA29. The host cytochrome oxidase (cox) gene was used as an internal reference to show that the peptide treatment had no effect on the amplification of cox. (A & B) Percentage clearance of the CLas by qPCR of DNA & RNA from the infected leaves. (C &D) Percentage clearance of the E. amylovora by qPCR of DNA & RNA from the infected leaves. RNA from the detached leaf assays described in FIG. 10 were used for gene expression analysis by qPCR to determine the effect of the treatment by heatmaps due to different peptides as shown on the innate immune genes. Details of the alteration of plant immune signaling pathways due to microbial infection are discussed in Front Microbiol 11, 1298.

(A) The selected citrus genes encode: Lipid-transfer protein 2, LTP2 (XM_006482145.3), Ethylene-responsive transcription factor 3, ERF003 (XM_006483296.3), Chitinase (XM_015532796.2), Zinc finger, C2H2 type (XM_015531045.2), GDSL esterase (XM_006478917.3), Abscisic acid induced-regulated protein (XM_025101123.1), LEA protein5, LEA5 (NM_001289140.1), Cytochrome P450 82G1 (XM_006479159.3), sodium/hydrogen exchanger 2 (XM_006479811.3), Phloem-specific lectin PP2-like protein (XM_025095878.1), Ethylene-responsive transcription factor 6, ERF006 (XM_006466962.3), Sweet sugar transporter 3 (XM_006490501.3), MAPK6 (XM_025097223.1), Defensein (XM_006470821.3), EDS (XM_006476627.2), Col1 (XM_006486308.3), MAPKK3 (XM_006470193.3), WRKY24 (XM_006468068.3), WRKY4 (XM_006483024.2), PR1 (XM_006474081.3), MYB13 (XM_006479482.2), PR2 (KAH9738797.1), PR3 (KDO71433.1). The selected genes were also shown by RNA-seq to be differentially expressed upon CLas infection in citrus. The expression of the selected genes in treated and untreated citrus leaves was normalized relative to the expression of the housekeeping GAPDH gene.

(B) The selected apple genes encode: Jaz17, JA receptor (MDP0000241358), bHLH, JA induced transcription factor (MDP0000242554), EBP, ethylene induced binding GCC element binding transcription factor (MDP0000241358), AP2/ERF: regulates the biosynthesis of carotenoid by regulating the transcription of PSY, PAL1, SA-inducing PHE ammonia lyase 1 (MDP0000388769) Chalcone and stilbene synthase in flavonoid synthesis (MDP0000168735), ribonuclease-like PR (MDP0000782085), Apple defensin (MDP0000362305), Acidic endochitinase-like protein (MDP0000280265), Intracellular Ras-group-related LRR protein (MDP0000281307), Chlorophyl binding protein PSII LHC (MDP0000708928), The light-harvesting complex, LHC (MDP0000601491), NAD(P)H dehydrogenase (MDP0000509613), Peroxidase super family (MDP243237), Sorbitol dehydrogenase, SDH-GroES-like zinc-binding alcohol dehydrogenase family protein (MDP0000515106), MDP0000850409, MDP0000364657, Gibberellic acid stimulated Arabidopsis (GASA) gene (MDP0000201700), Xyloglucan endotransglucosylases/hydrolases, XTH (MDP0000361876), Hydroxyproline-rich glycoprotein family protein (MDP0000248516), Fasciclin-like arabinogalactan-protein 7 (MDC015146.108:31720-32772), MDP0000297541, proline-rich receptor-like protein kinase (MDP0000511014), MDP0000268505, Nicotianamine synthase-like 4 (MDP0000412490), Hydroxyproline-rich glycoprotein family protein: MDP0000248516. The selected genes were also shown by RNA-seq to be differentially expressed upon E. amylovora infection in apple. The expression of the selected genes in treated and untreated apple leaves was normalized relative to the expression of the housekeeping GAPDH gene.

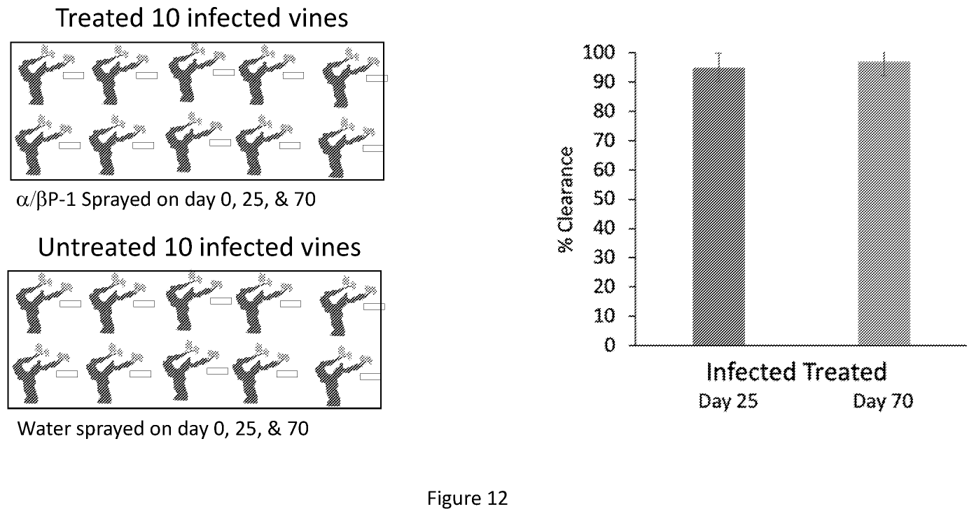

Preliminary field efficacy data to show that foliar spray of α/βP-1 clears Xf from the infected Chardonnay vines owned by Groth Vineyard & Winery, Napa, CA. The primer pair AAGGCAATAAACGCGCACTA (SEQ ID NO. 55) and HL6 reverse GGTTTTGCTGACTGGCAACA (SEQ ID NO. 56), was used for the qPCR of DNA extracted from untreated and treated leaves from the infected vines. Note that 25 mM α/βP-1 was sprayed on day 0, 25, and 70 and samples were analyzed for those days. As shown in FIG. 12, the clearance of Xf on day 25 and day 70 relative to day 0 (=untreated) was monitored. The treated and untreated blocks each had 10 infected vines. PR1 was chosen as the internal grape reference gene to show that the amplification of this gene remained unaltered in untreated vs. treated samples.

### Example 7: Materials and Methods

Phytotoxicity assays in plants: 10 μL of each peptide at different concentrations were infiltrated in leaves at different plants by a syringe. PBS Buffer was used as negative control. Two independent experiments were performed in which three leaves was inoculated in the abaxial/adaxial leaf at three different points. Necrotic effects were visually monitored to examine the possible toxicity of the peptide. The leaves were put on agar-plates (1%) for 7 days and maintained on controlled conditions at 26° C. and photoperiod of 16 h: 8 h (16 hours of light: 8 h of dark).

MIC: E. coli strain BL21 (5×10⁵ cfu) was inoculated for 16-20 hours at 37° C. with various concentrations of the peptide. The MIC was determined as the lowest concentration of the peptide that led to no visible growth of the bacteria. The final inoculum was diluted and plated on agar plates in parallel to the MIC incubation to confirm that the correct cfu was used in the determination of the MIC value.

Bioluminescence assay: The bioluminescence assay was conducted using the BacTiter-Glo Microbial Cell Viability Assay kit (Promega G8231). Bioluminescence was measured using plate reader. To determine exact CFU values, a standard curve was used to correlate the CFU to bioluminescence. Dose-response curves were obtained for most active peptides. To determine IC_{50%} and IC_{99%} values dose-response curves were fitted to Hill equation.

LIVE/DEAD cells staining: Overnight bacterial culture of E. coli BL21 was diluted in fresh LB 1:10 and bacterial growth continued for 2 h at 37° C. with aeration 200 rpm. 10 ml of bacterial culture were precipitated by centrifugation at 15 min, 5,000 rpm. Bacteria were re-suspended in 2 ml of 0.15 M NaCl. Three additional washes with 0.15 M NaCl were performed to remove traces of bacterial media. Bacterial concentration was adjusted with PBS to get a final concentration of 5×10⁶ cells per ml. The bacterial suspension was mixed with peptides solution in 0.15 M NaCl and incubated for 1 hour at room temperature. Cells were stained with LIVE/DEAD Bacterial Viability Kit (Thermo). Fluorescence of the live cells is green due to the SYTO™ 9 nucleic acid, SYTO 9 stain generally labels all bacteria in a population-those with intact membranes and those with damaged membrane. Fluorescence of the dead cells is red, since propidium iodide penetrates only bacteria with damaged membranes, causing a reduction in the SYTO 9 stain fluorescence when both dyes are present. For live cells control, cells were incubated with PBS only; for dead cells control, cells were killed with 70% isopropanol before staining.

LIVE/DEAD cells ratio was measured as described by manufacturer (Thermo). Cells were treated with peptides as described above. At the end of the incubation peptide-treated bacterial suspensions were mixed with equal volume of 2× working solution of the LIVE/DEAD dyes. Samples were incubated for 15 min at room temperature in the dark. Fluorescence intensity was measured by plate reader in black microtiter plates (Corning): Emission1, green: λex 485 nm, λem 530 nm, emission 2, red: λex 485 nm, λem 630 nm. Ratio was determined and normalized by control samples. thermofisher.com/order/catalog/product/L7012.

Hemolytic assay: Hemolytic assay was routinely performed using human erythrocytes. The procedure is based on measuring the hemoglobin release upon erythrocyte lysis. PBS pH 7.4 was used to suspend erythrocytes and dilute peptide samples. Human erythrocytes (RBC) were washed with PBS and adjusted to a concentration of 1% (v/v). 100 μl of 1% RBC was then mixed with 100 μl of testing samples, and the tubes were incubated at 37° C. for 60 min. Samples centrifuged for 5 min 14,000 g. Supernatants were collected and ODs 445 and 415 corresponding to the Soret bands of released hemoglobin were determined by Nanodrop. PBS and 0.01% Triton-X-100 were used respectively as negative (0%) and positive (100%) controls.

MTT Assay: The MTT assay is based on assessing cell metabolic activity, e.g., activity of NAD(P)H-dependent cellular oxidoreductases is proportional to the number of viable cells present. The oxidoreductases reduce the tetrazolium dye MTT (3-(4,5-dimethylthiazol-2-yl)-2,5-diphenyltetrazolium bromide) to insoluble purple dye, formazan. Cells were seeded at a density of 1× 10⁴ cells/well in 96-well culture plates and allowed to adhere overnight at 37° C. After 24 h of incubation, HEK294 cells were treated with 20 μM of peptides and incubated for 72 h. PBS, pH 7.4, the solvent used to make the peptides stock solution, was used as a no treatment control in this experiment. At 72 h post-stimulation, the cells were treated with 10 μL of 5 mg/mL MTT solution (Sigma-Aldrich) and incubated for another additional 3 h. 0.1% Triton X 100 was used as 100% control. The formazan crystals were dissolved in 100 μL of lysis solution, and the absorbance was determined at 570 nm using the microplate reader.

Detached leaf assay: Both uninfected and infected citrus (grapefruit) leaves were obtained from Kingsville, TX (curtsey: Kunta) and the infected and uninfected apple (red delicious) leaves were obtained from Las Cruces, NM (curtsey: Burke). Leaf samples were stored at −80° C. in sealed in Ziplock bags. Before the experiment, the Ziplock bags were put in box and transferred a −20° C. refrigerator. Before treatment, the leaves were thawed and dipped in the 1-2 ml peptide solution at the specified concentration at the room temperature for 48-96 hours in the biosafety cabinet. The leaves remained dipped till 0.8-1 ml peptide solution was absorbed. The leaves were then crushed inside the biosafety cabinet in liquid nitrogen using mortar and pestle. The crushed leaves were split into two halves: one for DNA extraction and the other for RNA as per instructions in (E.Z.N.A.® Plant DNA DS Kit; RNeasy plant mini kit). The extracted DNA and RNA were analyzed by qPCR in the BSL-1 lab. Forward and reverse primers for CLas detection are: GTCGAGCGCGTATGCAATACG (SEQ ID NO. 17) and CTACCTTTTTCTACGGGATAACGC (SEQ ID NO 18), which are chosen to amplify 16s DNA/RNA (27). Forward and reverse primers for E. amylovora detection are: CACTGATGGTGCCGTTG (SEQ ID NO. 19) and CGCCAGGATAGTCGCATA (SEQ ID NO. 20), which are chosen to amplify the locus in the plasmid pEA29 (28).

TABLE I
Minimum inhibitory concentrations(MIC in mM) single unit antibacterial peptides and
the chimeras constructed with the single antibacterial units.

| | Type of | | MIC |
|---|---|---|---|
| Code | Scaffold | Amino acid sequence | (mM) |
| 11P-1 | unit A | KKLIKKILKIL(SEQ ID. NO. 1) | 10-20 |
| | | KKLFKKILKYL*(SEQ ID. NO. 2) | |

| | | | |
|---|---|---|---|
| 11P-2 | unit B | FQWORNIRKVR**(SEQ ID. NO. 3) | >20 |
| 11P-3 | unit C | FWQRRIRRWRR(SEQ ID. NO. 4) | >20 |
| 8P-1 | unit D | RRWWRWWR(SEQ ID. NO. 5) | >20 |
| 16P-1 | unit E | IERSTNLDWYKGPTLL(SEQ ID. NO. 6) | >20 |
| Gen-1 | Chimera AA-1 | KKLPKEILKILGSGYGSLPKEILKILELKK(SEQ ID. NO. 21) | 5-10 |
| Gen-2 | Chimera AC-7 | KKLPEKILEILGSGYKKLPFWQRRIRRWRR(SEQ ID. NO. 22) | 2.5-5 |
| 30P-1 | Chimera AC-1 | KKLIKKILKILGSGYGSPGEWQRRIRRWRR(SEQ ID. NO. 23) | 1.25-2.5 |
| 30P-2 | Chimera CA-1 | FWQRRIRRWRRGSGYGSPGKKLIKKILKIL(SEQ ID. NO. 24) | 1.25-2.5 |
| 30P-3 | Chimera AC-2 | KKLPKKILKILGSGYGSPGEWQRRIRRWRR(SEQ ID. NO. 25) | 0.6-1.25 |
| 27P-1 | Chimera DC-1 | RRWWRWWRGSGYGSPGFWQRRIRRWRR(SEQ ID. NO. 26) | >20 |
| 27P-2 | Chimera CD-1 | FWQRRIRRWRRGSGYGSPGRRWWRWWR(SEQ ID. NO. 27) | 5-10 |
| 27P-3 | Chimera AC-3 | RKPARKVLKILGRGSEFWQKRVRRWRR(SEQ ID. NO. 28) | 10-20 |
| UGK-1 | Chimera CD-2 | RLPKAFQWORRLRRWRRPYSPGRRWWRWWR(SEQ ID. NO. 29) | 5-10 |
| UGK-5 | Chimera CD-3 | RLPEAFQWQRRLRRWRRPDSPGRRWWRWWR(SEQ ID. NO. 30) | 5-10 |
| UGK-9 | Chimera CD-4 | RLPEAFQWQRNIRKVRRPDSPGRRWWRWWR(SEQ ID. NO. 31) | 5-10 |
| UGK-13 | Chimera AC-3 | KKLPEKILKILESGYGSPGFWQRRIRRWRR(SEQ ID. NO. 32) | 0.625-1.25 |
| UGK-17 | Chimera AC-4 | KKLPEKILKILESLKGSPGFWQRRIRRWRR(SEQ ID. NO. 33) | 1.25-2.5 |
| UGK-21 | Chimera AC-5 | KKLPQKLLEILKSLKGSPGEWQRRIRRWRR(SEQ ID. NO. 34) | 1.25-2.5 |
| UGK-25 | Chimera AC-6 | KKLPEKLLEILKSLEGSPGFWQRRIRRWRR(SEQ ID. NO. 35) | 2.5-5 |
| UGK-29 | Chimera AD-1 | KPRGSEQLQELTRRLLDSPLERRWWEWMRR(SEQ ID. NO. 36) | >20 |
| UGK-33 | Chimera AD-2 | KPRLSEQLQELTRRLLDSPLERRWWEWWRR(SEQ ID. NO. 37) | >20 |
| UGK-37 | Chimera AD-3 | KPRGSEQLQELTRRLLDSPLERRFWOWMRR(SEQ ID. NO. 38) | >20 |
| UGK-41 | Chimera$ AD-4 | KRPEELLQKLKSLEGSKAHLQHHDWTSK(SEQ ID. NO. 39) | >20 |
| UGK-45 | Chimera$ AD-5 | LPKRLEELLQKLKSLEGSKAHEKLHDWTRK(SEQ ID. NO. 40) | >20 |
| UGK-49 | Chimera$ AD-6 | QPKRLEELLEKLKSLEGSKAHEKLHDWTRK(SEQ ID. NO. 41) | >20 |
| UGI-7 | Chimera DC-2 | RRWWRWWRGSYGSVYGSPGFWQRRIRRWRR(SEQ ID. NO. 42) | 5-10 |
| I27AB | Chimera E+1 | IERSRNLDWYKGPTLLDALKNLNEGKR(SEQ ID. NO. 43) | >20 |
| I31LB | Chimera E+2 | IERSRNLDWYKGPTLLDALKNLNEGKRPSDK(SEQ ID. NO. 44) | >20 |
| L19GA | S-S bridged#2 | KC2RRLC6YKQRC11VTYC15RGRQ@(SEQ ID. NO. 45) | 1.25-2.5 |

*Homolog of A: a hybrid of Cecropin and melittin; ** Similar to C; Intrachain disulfide(S-S) bridges C2-C15 & C6-C11; $: Distant homologs of AD; @: S-S bridged

TABLE II
Bactericidal activity of selected peptides
measured by bioluminescence assay

| | | IC_{99%}, μM | Hill |
|---|---|---|---|
| Peptide | IC_{50%}, μM | (~MIC) | coefficient |
| UGK-13/Chimera AC-3 | 1.24 ± 0.02 | 1.40 ± 0.03 | 36 |
| UGK-17/Chimera AC-4 | 1.36 ± 0.04 | 1.97 ± 0.06 | 12 |
| 30P-3/Chimera AC-2 | 1.45 ± 0.09 | 2.41 ± 0.11 | 9 |
| Gen-1/Chimera AA-1 | 10.22 ± 0.07 | 11.4 ± 0.07 | 41 |
| Gen-2/Chimera AC-7 | 2.83 ± 0.14 | 4.48 ± 0.22 | 10 |
| UGK-19GA/S-S | 1.49 ± 0.22 | 1.88 ± 0.29 | 20 |
| bridged#2 | | | |
| 11P-1/Unit A | 12.94 ± 0.62 | 29.38 ± 0.75 | 6 |
| 11P-3/Unit C | 48.98 ± 2.79 | 160.80 ± 3.25 | 4 |
| 11P-1 + 11P-3/Unit | 40.97 ± 1.11 | 60.00 ± 1.63 | 12 |
| A + Unit C | | | |

TABLE III
Measurement of toxicity of the selected chimeras on tobacco and tomato leaves

| | | | | | Volume | Toxicity |
|---|---|---|---|---|---|---|
| | | Dosage1 | Dosage2 | Dosage3 | Infiltrated | (monitored up to |
| Peptides | Control | (mM) | (mM) | (mM) | (mL) | 96 hours) |
| UGK-13 | water | 15 | 20 | 25 | 20 | Not toxic |
| UGK-17 | water | 15 | 20 | 25 | 20 | Not toxic |
| 30P-3 | water | 15 | 20 | 25 | 20 | Not toxic |
| UGK-9 | water | 15 | 20 | 25 | 20 | Not toxic |

TABLE IV
Data from the detached leaf assay

| | | |
|---|---|---|
| Treatment | DNA | RNA |

CLas clearance from infected citrus leaves

| | | |
|---|---|---|
| Water (control) | 23.99 ± 0.020 (9.4 × 10⁵ ± 0.051) | 26.04 ± 0.020 (7.4 × 10⁵ ± 0.071) |
| UGK-17 | 31.44 ± 0.013 (9.2 × 10³ ± 0.210) | 33.30 ± 0.026 (7.0 × 10² ± 0.310) |
| UGK-9 | 23.07 ± 0.009 (10.3 × 10⁵ ± 019) | 24.56 ± 0.019 (8.8 × 10⁵ ± 0.053) |
| UGK-13 | 28.05 ± 0.012 (5.5 × 10⁴ ± 0.065) | 31.12 ± 0.011 (7.7 × 10³ ± 0.065) |
| 30P-3 | 29.08 ± 0.018 (2.3 × 10⁴ ± 0.122) | 31.80 ± 0.028 (6.8 × 10³ ± 0.190) |

E. amylovora clearance from infected apple leaves

| | | |
|---|---|---|
| Water (control) | 28.73 ± 0.590 (5.610⁴ ± 0.07) | 28.91 ± 0.990 (5.4 × 10⁴ ± 0.051) |
| UGK-17 | 34.46 ± 0.028 (400 ± 0.31) | 34.10 ± 0.560 (700 ± 0.210) |
| UGK-9 | 35.66 ± 1.43 (~10²) | 32.62 ± 0.570 (4.7 × 10³ ± 0.019) |
| UGK-13 | 35.78 ± 2.98 (~10²) | 34.44 ± 0.620 (300 ± 0.065) |
| 30P-3 | 36.50 ± 0.89 (~10²) | 31.54 ± 0.018 (7.8 × 10³ ± 0.122) |

TABLE V
Bactericidal activity of exemplary peptides

| | | |
|---|---|---|
| Peptide | IC_{50%} (μM) | IC_{99%} (μM) |

ATCC

| | | |
|---|---|---|
| α/βP-1 | 1.4 ± 0.3 | 1.8 ± 0.3 |
| α/βP-2 | 1.2 ± 0.3 | 1.3 ± 0.3 |
| α/βP-3 | 0.7 ± 0.2 | 1.0 ± 0.2 |

BL21

| | | |
|---|---|---|
| α/βP-1 | 1.2 ± 0.1 | 1.4 ± 0.1 |
| α/βP-2 | 1.0 ± 0.3 | 1.6 ± 0.3 |
| α/βP-3 | 2.2 ± 0.8 | 2.6 ± 1.0 |

TABLE sIa
Citrus homologs
Alignment statistics for the chimeras AC
1. N-terminal fragment
AMP N domain-containing protein [Citrus sinensis]; GenBank: KAH9762819.1

| Score | Expect | Identities | Positives | Gaps |
|---|---|---|---|---|
| 31.6 bits(67) | 0.26 | 11/14(79%) | 11/14(78%) | 2/14(14%) |

Query 2 KLPEKILKILESLK 15
KL EKI ILESLK
Sbjct 332 KLAEKI--ILESLK 343
2. Middle part
putative disease resistance protein RGA3 [Citrus clementina];
NCBI Reference Sequence: XP_024036637.1

| Score | Expect | Identities | Positives | Gaps |
|---|---|---|---|---|
| 26.5 bits(55) | 14 | 8/8(100%) | 8/8(100%) | 0/8(0%) |

Query 10 ILESLKGS 17
ILESLKGS
Sbjct 267 ILESLKGS 274
3. 25/30 amino acid length of the chimeras AC
Extra-large guanine nucleotide-binding protein 3
[Citrus sinensis]; GenBank: KAH9687262.1

| Score | Expect | Identities | Positives | Gaps |
|---|---|---|---|---|
| 30.3 bits(64) | 0.71 | 13/25(52%) | 16/25(64%) | 7/25(28%) |

Query 1 KKLPEKILKILESLKGSPGFWQRRI 25
KKL EK +K L+SLK QRR+
Sbjct 503 KKL-EKLMKQLQSLK------QRRV 520
4. C-terminal part
protein kinase domain-containing protein [Citrus sinensis]; GenBank: KAH9801395.1

| Score | Expect | Identities | Positives | Gaps |
|---|---|---|---|---|
| 25.2 bits(52) | 38 | 7/8(88%) | 7/8(87%) | 1/8(12%) |

Query 20 FWQRRIRR 27
FWQ RIRR
Sbjct 695 FWQ-RIRR 701
B. Alignment statistics for the chimeras CA
1. 1. N-terminal plus linker
Valin--tRNA ligase 1 [Citrus sinensis]; GenBank: KAH9646357.1

| Score | Expect | Identities | Positives | Gaps |
|---|---|---|---|---|
| 26.1 bits(54) | 20 | 8/10(80%) | 8/10(80%) | 0/10(0%) |

Query 6 IRRWRRGSGY 15
I RWRR SGY
Sbjct 290 IIRWRRMSGY 299
hypothetical protein CISIN_1g0401331mg, partial
[Citrus sinensis]; GenBank: KDO48896.1

| Score | Expect | Identities | Positives | Gaps |
|---|---|---|---|---|
| 26.9 bits(56) | 10 | 10/17(59%) | 10/17(58%) | 4/17(23%) |

```
Query 1 FW---QRRIRRWRRGSG 14
                    FW Q R RR R GSG
  Sbjct 816 FWRIRQKR-RRRRKGSG 831
2. 2. C-terminal part
peptidyl-prolyl cis-trans isomerase FKBP62
[Citrus sinensis]; GenBank: KAH9770943.1
```

| Score | Expect | Identities | Positives | Gaps |
|---|---|---|---|---|
| 26.9 bits(56) | 10 | 8/9(89%) | 8/9(88%) | 0/9(0%) |

Query 20 KKLIKKILK 28
KK IKKILK
Sbjct 270 KKVIKKILK 278
3. 3. Linker plus the C-terminal part
hypothetical protein CUMW_263420 [Citrus unshiu]; GenBank: GAY68349.1

| Score | Expect | Identities | Positives | Gaps |
|---|---|---|---|---|
| 25.7 bits(53) | 26 | 8/11(73%) | 9/11(81%) | 0/11(0%) |

Query 17 SPGKKLIKKIL 27
SP KKL+KK L
Sbjct 11 SPNKKLVKKFL 21
4. 4. Linker
hypothetical protein CISIN_1g0415571mg,
partial [Citrus sinensis]; GenBank: KDO48753.1

| Score | Expect | Identities | Positives | Gaps |
|---|---|---|---|---|
| 26.9 bits(56) | 9.1 | 8/9(89%) | 8/9(88%) | 0/9(0%) |

Query 10 RRGSGYGSP 18
RRGS YGSP
Sbjct 32 RRGSSYGSP 40
C. Alignment statistics for the chimeras CD
1. C-terminal
protein DETOXIFICATION 19 isoform X3
[Citrus clementina]; Sequence ID: XP_024033441.1

| Score | Expect | Identities | Positives | Gaps |
|---|---|---|---|---|
| 26.5 bits(55) | 14 | 6/7(86%) | 6/7(85%) | 0/7(0%) |

Query 23 RRWWRWW 29
RRWWR W
Sbjct 31 RRWWRNW 37
2. Linker plus C-terminal
histone deacetylase 15 isoform X1 [Citrus sinensis]
Sequence ID: XP_006485755.1

| Score | Expect | Identities | Positives | Gaps |
|---|---|---|---|---|
| 32.9 bits(70) | 0.095 | 9/17(53%) | 10/17(58%) | 2/17(11%) |

Query 12 IRKVRRPDSPGRRWWRW 28
I K+RR D P WW W
Sbjct 581 IKKIRRADAP--IWWKW 595
3. N-terminal plus linker
Subtilisin-like protease SBT6.1 [Citrus clementina];
Sequence ID: XP_006451169.

| Score | Expect | Identities | Positives | Gaps |
|---|---|---|---|---|
| 26.9 bits(56) | 7.8 | 10/17(59%) | 10/17(58%) | 4/17(23%) |

Query 1 FW--QRRIRRWRRGSG 14
FW Q R RR R GSG
Sbjct 1008 FWRIRQKR-RRRRKGSG 1023
D. Alignment statistics for the chimeras DC
1. 1. N terminal
protein DETOXIFICATION 19 isoform X1 [Citrus clementina];
NCBI Reference Sequence: XP_024033440.1

| Score | Expect | Identities | Positives | Gaps |
|---|---|---|---|---|
| 28.2 bits(59) | 2.8 | 7/8(88%) | 7/8(87%) | 0/8(0%) |

Query 1 RRWWRWWR 8
RRWWR WR
Sbjct 33 RRWWRKWR 40
2. Middle part(with linker)
Flowering locus K domain [Citrus sinensis]
Sequence ID: KAH9699095.1

| Score | Expect | Identities | Positives | Gaps |
|---|---|---|---|---|
| 26.5 bits(55) | 14 | 8/9(89%) | 8/9(88%) | 0/9(0%) |

Query 9 GSYGSVYGS 17
G YGSVYGS
Sbjct 513 GGYGSVYGS 521
3. Linker plus C-terminal chaperone protein ClpB1 [Citrus sinensis]
Sequence ID: XP_006477653.

| Score | Expect | Identities | Positives | Gaps |
|---|---|---|---|---|
| 26.9 bits(56) | 10 | 9/19(47%) | 10/19(52%) | 5/19(26%) |

Query 10 SYGSVYGSPGFWQRRIRRW 28
SY +YG R IRRW
Sbjct 809 SYDPIYGA-----RPIRRW 822
4. E. Alignment for the S-S bridged L19GA
1. 13 out of 19
Endonuclease [Citrus sinensis]; Sequence ID: KAH9697901.

| Score | Expect | Identities | Positives | Gaps |
|---|---|---|---|---|
| 23.1 bits(47) | 67 | 9/13(69%) | 9/13(69%) | 3/13(23%) |

Query 4 RLCYKQRCVTYCR 16
RLC RCVT CR
Sbjct 681 RLC--GRCVT-CR 690
2. N-terminal
Cyclic nucleotide-gated ion channel 1
[Citrus sinensis]; Sequence ID: KAH9646573.1

| Score | Expect | Identities | Positives | Gaps |
|---|---|---|---|---|
| 27.8 bits(58) | 1.5 | 7/7(100%) | 7/7(100%) | 0/7(0%) |

Query 3 RRLCYKQ 9
RRLCYKQ
Sbjct 517 RRLCYKQ 523
3. C-terminal
Timeless family protein [Citrus sinensis]; Sequence ID: KAH9677436.1

| Score | Expect | Identities | Positives | Gaps |
|---|---|---|---|---|
| 23.1 bits(47) | 67 | 6/7(86%) | 6/7(85%) | 0/7(0%) |

Query 9 QRCVTYC 15
QRCV YC
Sbjct 40 QRCVEYC 46

TABLE sIb
Apple homologs
A. Alignment statistics for the chimeras AC
1. N-terminal plus linker
hypothetical protein DVH24_018081 [Malus domestica]; GenBank: RXI06039.1

| Score | Expect | Identities | Positives | Gaps |
|---|---|---|---|---|
| 29.9 bits(63) | 0.26 | 12/17(71%) | 13/17(76%) | 3/17(17%) |

Query 2 KLPEKILKILESLK-GS 17
KL EK+ ILESLK GS
Sbjct 304 KLAEKV--ILESLKRGS 318
2. 22 out of 30 residues
hypothetical protein DVH24_018947 [Malus domestica]; GenBank: RXH71592.1

| Score | Expect | Identities | Positives | Gaps |
|---|---|---|---|---|
| 25.2 bits(52) | 9.8 | 13/25(52%) | 14/25(56%) | 7/25(28%) |

Query 5 EKILK--ILE-SLKGSPGFWQRRIR 26
+K LK ILE L GSPG RR
Sbjct 288 DKELKWHILEIPLNGSPG----RLR 308
3. C-terminal
dnaJ protein ERDJ3B [Malus domestica];
NCBI Reference Sequence: XP_008386734.2

| Score | Expect | Identities | Positives | Gaps |
|---|---|---|---|---|
| 25.2 bits(52) | 9.9 | 7/10(70%) | 7/10(70%) | 0/10(0%) |

Query 21 WQRRIRRWRR 30
W RR RR RR
Sbjct 113 WRRRRRRRRR 122
protein SUPPRESSOR OF npr1-1, CONSTITUTIVE 1-like [Malus domestica];
NCBI Reference Sequence: XP_008382508.3

| Score | Expect | Identities | Positives | Gaps |
|---|---|---|---|---|
| 25.7 bits(53) | 7.1 | 7/10(70%) | 7/10(70%) | 0/10(0%) |

Query 19 GFWQRRIRRW 28
GFWQR R W
Sbjct 612 GFWQRQQRWW 621
B. Alignment statistics for the chimeras CA
1. N-terminal plus linker
LOW QUALITY PROTEIN: subtilisin-like protease SBT6.1 [Malus domestica]
Sequence ID: XP_008361242.

| Score | Expect | Identities | Positives | Gaps |
|---|---|---|---|---|
| 26.9 bits(56) | 2.7 | 10/17(59%) | 10/17(58%) | 4/17(23%) |

Query 1 FW---QRRIRRWRRGSG 14
FW Q R RR R GSG
Sbjct 1021 FWRIRQKR-RRRRKGSG 1036
2. Linker plus C-terminal
probable E3 ubiquitin-protein ligase BAH1-like 1 [Malus domestica]
Sequence ID: XP_028954001.

| Score | Expect | Identities | Positives | Gaps |
|---|---|---|---|---|
| 24.0 bits(49) | 26 | 10/14(71%) | 10/14(71%) | 4/14(28%) |

Query 18 PG---KKLIKKILK 28
PG KKL KKILK
Sbjct 19 PGVGFKKL-KKILK 31
TMV resistance protein N-like [Malus domestica]; Sequence ID: XP_028945095.

| Score | Expect | Identities | Positives | Gaps |
|---|---|---|---|---|
| 23.1 bits(47) | 52 | 13/25(52%) | 13/25(52%) | 9/25(36%) |

Query 13 SGYGSPGKK------LIKKIL-KIL 30
SGY P KK LI KI KIL
Sbjct 194 SGY--PLKKEDSEATLINKIVKKIL 216
C. Alignment statistics for the chimeras CD
1. N-terminal
hypothetical protein DVH24_024111 [Malus domestica]
Sequence ID: RXH94427.1

| Score | Expect | Identities | Positives | Gaps |
|---|---|---|---|---|
| 25.2 bits(52) | 7.8 | 7/10(70%) | 7/10(70%) | 0/10(0%) |

Query 2 WQRRIRRWRR 11
W RR RR RR
Sbjct 303 WRRRRRRRRR 312
2. N-terminal plus linker
Subtilisin-like protease SBT6.1 [Malus domestica]; Sequence ID: XP_008361242.

| Score | Expect | Identities | Positives | Gaps |
|---|---|---|---|---|
| 26.9 bits(56) | 2.0 | 10/17(59%) | 10/17(58%) | 4/17(23%) |

Query 1 FW---QRRIRRWRRGSG 14
FW Q R RR R GSG
Sbjct 1021 FWRIRQKR-RRRRKGSG 1036
3. The whole length
late embryogenesis abundant protein M17-like [Malus domestica]
Sequence ID: XP_028963845.

| Score | Expect | Identities | Positives | Gaps |
|---|---|---|---|---|
| 35.0 bits(75) | 0.003 | 16/36(44%) | 16/36(44%) | 15/36(41%) |

Query 2 WQR------R-IR-RWRRGSGYGSPGRR--WWRWWR 27
WQR R R RW RG GRR WRW R
Sbjct 160 WQRWRGQGRRGTRWRWKRG-----QGRRGTRWRWRR 190
4. Linker plus C-Terminal
uncharacterized protein LOC103424581 [Malus domestica];
Sequence ID: XP 008360894.

| Score | Expect | Identities | Positives | Gaps |
|---|---|---|---|---|
| 24.4 bits(50) | 15 | 8/16(50%) | 9/16(56%) | 1/16(6%) |

Query 11 RGSGYGSPGRRWWRWW 26
R S +GRR W WW
Sbjct 123 RASSFSKHGRR-WSWW 137
D. Alignment statistics for the chimeras DC
1. N-terminal
protein DETOXIFICATION 19 [Citrus sinensis]; Sequence ID: KAH9802589.1

| Score | Expect | Identities | Positives | Gaps |
|---|---|---|---|---|
| 28.2 bits(59) | 3.7 | 7/8(88%) | 7/8(87%) | 0/8(0%) |

Query 1 RRWWRWWR 8
RRWWR WR
Sbjct 33 RRWWRKWR 40
2. Linker plus C-terminal
chaperone protein ClpB1 [Citrus sinensis]; Sequence ID: XP_006477653.1

| Score | Expect | Identities | Positives | Gaps |
|---|---|---|---|---|
| 26.9 bits(56) | 10 | 9/19(47%) | 10/19(52%) | 5/19(26%) |

Query 10 SYGSVYGSPGFWQRRIRRW 28
SY +YG R IRRW
Sbjct 809 SYDPIYGA-----RPIRRW 822
E. Alignment statistics for the S-S bridged L19GA
1. N-terminal aspartyl protease family protein 2-like
[Malus domestica]; Sequence ID: XP_008343434.2

| Score | Expect | Identities | Positives | Gaps |
|---|---|---|---|---|
| 24.8 bits(51) | 4.4 | 7/8(88%) | 7/8(87%) | 1/8(12%) |

Query 2 CRRLCYKQ 9
CRR CYKQ
Sbjct 175 CRR-CYKQ 181
2. C-terminal
peamaclein [Malus domestica]; Sequence ID: XP_008360977.1

| Score | Expect | Identities | Positives | Gaps |
|---|---|---|---|---|
| 24.8 bits(51) | 4.2 | 6/9(67%) | 7/9(77%) | 0/9(0%) |

Query 7 YKQRCVTYC 15
YK+RC YC
Sbjct 43 YKERCLKYC 51
3. 16 out of 19
probable plastid-lipid-associated protein 13, chloroplastic
isoform X2 [Malus domestica] Sequence ID: XP_008392074.2

| Score | Expect | Identities | Positives | Gaps |
|---|---|---|---|---|
| 24.4 bits(50) | 6.2 | 10/21(48%) | 10/21(47%) | 8/21(38%) |

Query 2 CRRLCYK------QRCVTYCR 16
CRR CY QR V CR
Sbjct 46 CRRKCYRDGRISFQRSV--CR 64

TABLE sIIa
CLas clearance by different chimeras by detached leaf assays

| | DNA | | | | | | | | | |
|---|---|---|---|---|---|---|---|---|---|---|
| | HLBas (CLas primer) | | | | | | | | | |
| | | HLBas (CLas primer) | | | | | | | | |
| | healthy | Infected | UGK-17-25 | UGK-17-20 | UGK-9-25 | UGK-9-20 | UGK13-25 | UGK-13-20 | 30P-3-20 | 30P-3-25 |
| | 34.57 | 23.65 | 31.75 | 28.62 | 22.93 | 24.93 | 27.8 | 24.78 | 26.45 | 28.06 |
| | 36.17 | 24.34 | 30.74 | 29.94 | 23.21 | 24.26 | 28.3 | 25.09 | 27 | 27 |
| Mean | 35.37 | 23.995 | 31.245 | 29.28 | 23.07 | 24.595 | 28.05 | 24.935 | 26.725 | 27.53 |
| SD | 1.131 | 0.488 | 0.714 | 0.933 | 0.198 | 0.474 | 0.354 | 0.219 | 0.389 | 0.750 |
| CV | 0.032 | 0.020 | 0.023 | 0.032 | 0.009 | 0.019 | 0.013 | 0.009 | 0.015 | 0.027 |

COX primer

| | | | | | | | | | | |
|---|---|---|---|---|---|---|---|---|---|---|
| | 16.14 | 15.61 | 20.72 | 19.18 | 16.24 | 17.72 | 18.85 | 17.94 | 16.34 | 15.81 |
| | 16.1 | 16.01 | 19.52 | 18.45 | 16.12 | 18.29 | 19.54 | 16.22 | 15.5 | 16.15 |
| Mean | 16.12 | 15.81 | 20.12 | 18.815 | 16.18 | 18.005 | 19.195 | 17.08 | 15.92 | 15.98 |
| SD | 0.028 | 0.283 | 0.849 | 0.516 | 0.085 | 0.403 | 0.488 | 1.216 | 0.594 | 0.240 |
| CV | 0.002 | 0.018 | 0.042 | 0.027 | 0.005 | 0.022 | 0.025 | 0.071 | 0.037 | 0.015 |

RNA
HLBas (CLas primer)

Peptide: 25 μM

| | Healthy | Infected | UGK-17 | UGK-13 | UGK-9 | 30P-3 |
|---|---|---|---|---|---|---|
| | 40 | 26.41 | 33.36 | 30.88 | 26.97 | 29.94 |
| | 36.77 | 25.67 | 32.15 | 31.35 | 27.22 | 30.37 |
| Mean | 38.385 | 26.04 | 32.755 | 31.115 | 27.095 | 30.155 |
| SD | 2.284 | 0.523 | 0.856 | 0.332 | 0.177 | 0.304 |
| CV | 0.060 | 0.020 | 0.026 | 0.011 | 0.007 | 0.010 |

GAPDH primer

| | | | | | | |
|---|---|---|---|---|---|---|
| | 26.67 | 24.19 | 26.44 | 24.78 | 24.67 | 25.61 |
| | 25.08 | 25.5 | 26.42 | 24.73 | 23.63 | 23.66 |
| Mean | 25.875 | 24.845 | 26.43 | 24.755 | 24.15 | 24.635 |
| SD | 1.124 | 0.926 | 0.014 | 0.035 | 0.735 | 1.379 |
| CV | 0.043 | 0.037 | 0.001 | 0.001 | 0.030 | 0.056 |

TABLE sIIb
E. amylovora clearance by different chimeras by detached leaf assays
DNA
Erwinia
Primer
(P29T)

| | | | | | | | | | Infected- |
|---|---|---|---|---|---|---|---|---|---|
| | UKG-9-20 | UKG-9-25 | UKG-13-20 | UKG-13-25 | UKG-17-20 | UKG-17-25 | 30P-3-20 | 30P-3-25 | water |
| | 37.72 | 37.69 | 36.21 | 40 | 38.4 | 34.5 | 37.67 | 37.76 | 29.56 |
| | 36.52 | 33.63 | 37.19 | 31.56 | 32.78 | 34.42 | 36.71 | 35.24 | 27.89 |
| Mean | 37.12 | 35.66 | 36.7 | 35.78 | 35.59 | 34.46 | 37.19 | 36.5 | 28.725 |
| SD | 0.8485 | 2.8709 | 0.693 | 5.968 | 3.9739 | 0.0566 | 0.6788 | 1.7819 | 1.1809 |
| SE | 0.4243 | 1.4354 | 0.3465 | 2.984 | 1.987 | 0.0283 | 0.3394 | 0.891 | 0.5904 |
| CV | 0.0229 | 0.0805 | 0.0189 | 0.1668 | 0.1117 | 0.0016 | 0.0183 | 0.0488 | 0.0411 |
| | Apple Primer (UBI) | | | | | | | | |
| | 24.42 | 25.36 | 20.73 | 20.05 | 22.47 | 23.21 | 21.67 | 24.45 | 24.78 |
| | 22.11 | 22.37 | 21.45 | 21.6 | 23.93 | 22.26 | 22.46 | 26.75 | 25.88 |
| Mean | 23.265 | 23.865 | 21.09 | 20.825 | 23.2 | 22.735 | 22.065 | 25.6 | 25.33 |
| SD | 1.633 | 2.114 | 0.509 | 1.096 | 1.032 | 0.672 | 0.559 | 1.626 | 0.778 |
| SE | 0.817 | 1.057 | 0.255 | 0.548 | 0.516 | 0.336 | 0.279 | 0.813 | 0.389 |
| CV | 0.07 | 0.089 | 0.024 | 0.053 | 0.044 | 0.03 | 0.025 | 0.064 | 0.031 |

RNA
Erwinia
Primer
(P29T)

| | 25∞M | | | | Infected- |
|---|---|---|---|---|---|
| Peptide | UGK-17 | UGK-9 | UGK-13 | 30P-3 | water |
| | 34.89 | 33.42 | 33.56 | 31.56 | 29.61 |
| | 33.31 | 31.81 | 35.31 | 31.51 | 28.21 |
| Mean | 34.1 | 32.615 | 34.435 | 31.535 | 28.91 |
| SD | 1.11722871 | 1.13844192 | 1.23743687 | 0.03535534 | 0.989949494 |
| SE | 0.55861436 | 0.56922096 | 0.61871843 | 0.01767767 | 0.494974747 |
| CV | 0.03276331 | 0.03490547 | 0.03593544 | 0.00112115 | 0.034242459 |

Apple
GAPDH

| | | | | | |
|---|---|---|---|---|---|
| | 25.94 | 25.57 | 25.94 | 25.94 | 25.57 |
| | 24.09 | 26.84 | 24.09 | 24.09 | 26.84 |
| Mean | 25.015 | 26.205 | 25.015 | 25.015 | 26.205 |
| SD | 1.30814755 | 0.89802561 | 1.30814755 | 1.30814755 | 0.898025612 |
| SE | 0.65407377 | 0.44901281 | 0.65407377 | 0.65407377 | 0.449012806 |
| CV | 0.05229453 | 0.03426925 | 0.05229453 | 0.05229453 | 0.034269247 |

### REFERENCES

1. Hu B, Rao M J, Deng X, Pandey S S, Hendrich C, Ding F, et al. 2021. Molecular signatures between citrus and Candidatus Liberibacter asiaticus. PLoS Pathog 17 (12): e1010071.
2. da Graça J V, Korsten L. 2004. Citrus Huanglongbing: Review, Present status and Future Strategies. In: Naqvi S.A.M.H. (eds) Diseases of Fruits and Vegetables Volume I. Springer, Dordrecht.
3. Graham J, Gottwald T, Setamou M. 2020. Status of Huanglongbing (HLB) outbreaks in Florida, California, and Texas. Tropical Plant Pathology 46:265-278.
4. Rehberg R A, Trivedi P, Bahureksa W, Sharp J L, Stokes S C, Menger R F, Borch T. 2021. Quantification of insecticide spatial distribution within individual citrus trees and efficacy through Asian citrus psyllid reductions under different application methods. Pest Manag Sci. 77 (4): 1748-1756.
5. Boina D R, Bloomquist J R. 2015. Chemical control of the Asian citrus psyllid and of huanglongbing disease in citrus. Pest Manag Sci. 71 (6): 808-23.
6. Tansey J A, Vanaclocha P, Monzo C, Jones M, Stansly P A. Pest Manag Sci. 2017 May; 73 (5): 904-916.
7. Johnson K B and Stockwell V O. 1998. Management of fire blight: A case study in microbial ecology. Annu. Rev. Phytopathol. 36:227-248.
8. Hong-Geller E, Möllhoff M, Shiflett P R, Gupta G. 2004. Design of chimeric receptor mimics with different TcRVbeta isoforms. Type-specific inhibition of superantigen pathogenesis. J Biol Chem. 279 (7): 5676-5684.
9. Lehnert N M, Allen D L, Allen B L, Catasti P, Shiflett P R, Chen M, Lehnert B E, Gupta G. 2001. Structure-based design of a bispecific receptor mimic that inhibits T cell responses to a superantigen. Biochemistry. 40 (14): 4222-4228.
10. Vuyisich M, Gnanakaran S, Lovchik J A, Lyons C R, Gupta G. 2008. A dual-purpose protein ligand for effective therapy and sensitive diagnosis of anthrax. Protein J. 27 (5): 292-302.
11. Kunkel M, Vuyisich M, Gnanakaran G, Bruening G E, Dandekar A M, Civerolo E, Marchalonis J J, Gupta G. 2007. Rapid clearance of bacteria and their toxins: development of therapeutic proteins. Crit Rev Immunol. 27 (3): 233-245.
12. Ali S, Ganai B A, Kamili A N, Bhat A A, Mir Z A, Bhat J A, Tyagi A, Islam S T, Mushtaq M, Yadav P, Rawat S, Grover A. 2018. Pathogenesis-related proteins and peptides as promising tools for engineering plants with multiple stress tolerance. Microbiol Res. 212-213:29-37.
13. Jones J D, Dangl J L. 2006. The plant immune system. Nature. 444 (7117): 323-329.
14. COMPOSITIONS AND METHODS FOR THE TREATMENT OF PATHOGENIC INFECTIONS IN PLANTS. Inventors: GUPTA, Goutam, STOVER, Eddie W. A US Patent. Publication Number: WO/2020/072535. Publication Date: 9 Apr. 2020.
15. Li J, Hu S, Jian W, Xie C, Yang X. 2021. Plant antimicrobial peptides: structures, functions, and applications. Bot Stud. 62 (1): 5.
16. Cunsolo V, Schicchi R, Chiaramonte M, Inguglia L, Arizza V, Cusimano M G, Schillaci D, Di Francesco A, Saletti R, Lo Celso F, Barone G, Vitale M. 2020. Identification of New Antimicrobial Peptides from Mediterranean Medical Plant Charybdis pancration (Steinh.) Speta. Antibiotics (Basel). 9 (11): 747.
17. Huan Y, Kong Q, Mou H, Yi H. 2020. Antimicrobial Peptides: Classification, Design, Application and Research Progress in Multiple Fields. Front Microbiol. 11:582779.
18. Dos Santos-Silva C A, Zupin L, Oliveira-Lima M, Vilela L M B, Bezerra-Neto J P, Ferreira-Neto J R, Ferreira J D C, de Oliveira-Silva R L, Pires C J, Aburjaile F F, de Oliveira M F, Kido E A, Crovella S, Benko-Iseppon A M. 2020. Plant Antimicrobial Peptides: State of the Art, In Silico Prediction and Perspectives in the Omics Era. Bioinform Biol Insights. 14:1177932220952739.
19. Waterhouse A, Bertoni M, Bienert S, Studer G, Tauriello G, Gumienny R, Heer F T, de Beer T A P, Rempfer C, Bordoli L, Lepore R, Schwede T. 2018. SWISS-MODEL: homology modelling of protein structures and complexes. Nucleic Acids Res. 46 (W1): W296-W303.
20. van Gunsteren W F, Bakowies D, Baron R, Chandrasekhar I, Christen M, Daura X, Gee P, Geerke D P, Glättli A, Hünenberger P H, Kastenholz M A, Oostenbrink C, Schenk M, Trzesniak D, van der Vegt N F, Yu H B. 2006. Biomolecular modeling: Goals, problems, perspectives. Angew Chem Int Ed Engl. 45 (25): 4064-4092.
21. Wiegand I, Hilpert K, Hancock R. 2008. Agar and broth dilution methods to determine the minimal inhibitory concentration (MIC) of antimicrobial substances. Nat Protoc. 3:163-175.
22. Paciello L, Falco F C, Landi C, Parascandola P. 2013. Strengths and weaknesses in the determination of Saccharomyces cerevisiae cell viability by ATP-based bioluminescence assay. Enzyme Microb Technol. 52 (3): 157-162.
23. Chen J, Wang X, Wang S, Chen C, Zhang W, Zhang Y. 2021. Ultra-Rapid Drug Susceptibility Testing for Klebsiella pneumoniae Clinical Isolates in 60 Min by SYBR Green I/Propidium Iodide Viability Assay. Front Microbiol. 12:694522.
24. Inui Kishi R N, Stach-Machado D, Singulani J D L, Dos Santos C T, Fusco-Almeida A M, Cilli, E M, Freitas-Astúa, J, Picchi, S C, Machado, M A. 2018. Evaluation of cytotoxicity features of antimicrobial peptides with potential to control bacterial diseases of citrus. PLOS One, 13 (9): p.e0203451.
25. Sineva E V, Andreeva-Kovalevskaya Z I, Shadrin A M, Gerasimov Y L, Ternovsky V I, Teplova V V, Solonin A S. (2009). Expression of Bacillus cereus hemolysin II in Bacillus subtilis renders the bacteria pathogenic for the crustacean Daphnia magna. FEMS microbiology letters, 299 (1): 110-119.
26 Denizot F, Lang R. 1986. Rapid colorimetric assay for cell growth and survival: modifications to the tetrazolium dye procedure giving improved sensitivity and reliability. Journal of immunological methods 89 (2): 271-277.
27 Bao M, Zheng Z, Sun X, Chen J, Deng X. 2020. Enhancing PCR Capacity to Detect Candidatus Liberibacter asiaticus' Utilizing Whole Genome Sequence Information Plant Dis. 104 (2): 527-532.
28. Salm H, Geider. 2004. Real-time PCR for detection and quantification of Erwinia amylovora, the causal agent of fire blight. Plant Pathology. 53 (4): 602610.
29 Nishad R, Ahmed T, Rahman V J, Kareem A. 2020. Modulation of Plant Defense System in Response to Microbial Interactions. Front Microbiol. 11:1298.
30 Yuan M, Ngou B P M, Ding P, Xin X F. 2021. PTI-ETI crosstalk: an integrative view of plant immunity. Curr Opin Plant Biol. 62:102030.
31 Bakshi M, Oelmüller R. 2014. WRKY transcription factors: Jack of many trades in plants. Plant Signal Behav. 9 (2): e27700.
32 Zhang W, Zhao F, Jiang L, Chen C, Wu L, Liu Z. 2018. Different Pathogen Defense Strategies in Arabidopsis: More than Pathogen Recognition. Cells. 7 (12): 252.
33 Li N, Han X, Feng D, Yuan D, Huang L J. 2019. Signaling Crosstalk between Salicylic Acid and Ethylene/Jasmonate in Plant Defense: Do We Understand What They Are Whispering? Int J Mol Sci. 20 (3): 671.
34 Wang Y, Zhou L, Yu X, Stover E, Luo F, Duan Y. 2016. Transcriptome Profiling of Huanglongbing (HLB) Tolerant and Susceptible Citrus Plants Reveals the Role of Basal Resistance in HLB Tolerance. Front Plant Sci. 7:933.
35. Kamber T, Buchmann J P, Pothier J F, Smits T H, Wicker T, Duffy B. 2016. Fire blight disease reactome: RNA-seq transcriptional profile of apple host plant defense responses to Erwinia amylovora pathogen infection. Sci Rep. 6:21600.
36 Joo H S, Fu C I, Otto M. 2016. Bacterial strategies of resistance to antimicrobial peptides. Philos Trans R Soc Lond B Biol Sci. 371 (1695): 20150292.

1. A composition having a first α/β peptide domain and a second α/β peptide domain coupled by a linker domain forming an antimicrobial chimeric peptide sequence, wherein the first and second α/β peptide domain are not homologous.

2-4. (canceled)

5. The composition of claim 1, wherein said first α/β peptide domain and/or said second α/β peptide domain are each selected from the group consisting of: a unit A peptide, a unit C/B peptide, a unit D peptide, and a unit E peptide.

6. The composition of claim 1, wherein said first α/β peptide domain and/or said second α/β peptide domain are each selected from the group consisting of: SEQ ID NO.'s 1-6, 57, or a variant or homolog thereof.

7. The composition of claim 1, wherein said first α/β peptide domain comprises a unit A peptide and said second α/β peptide domain is a C/B peptide, or a unit D peptide.

8. The composition of claim 7, wherein said chimeric peptide sequence is selected from the group consisting of: SEQ ID NO.'s 21-45, and 54, or a fragment or variant thereof.

9. (canceled)

10. The composition of claim 1, wherein said first α/β peptide comprises the peptide having an amino acid sequence according to SEQ ID NO.'s 44-52, or a fragment or variant thereof, and/or wherein said second α/β peptide comprises the peptide having an amino acid sequence according to SEQ ID NO.'s 53 or 57, or a fragment or variant thereof.

11-21. (canceled)

22. A composition for increasing the innate immune response in a plant infected with, or at risk of being infected with a pathogen comprising a first α/β peptide domain and a second α/β peptide domain coupled by a linker domain forming an antimicrobial chimeric peptide sequence, wherein the first and second α/β peptide domain are not homologous.

23. (canceled)

24. The composition of claim 22, wherein said first α/β peptide domain comprises a variant or homolog of said first α/β peptide domain, and wherein said second α/β peptide domain comprises a variant or homolog of said second α/β peptide domain.

25. (canceled)

26. The composition of claim 22, wherein said first α/β peptide domain and/or said second α/β peptide domain are each selected from the group consisting of: a unit A peptide, a unit C/B peptide, a unit D peptide, and a unit E peptide.

27. The composition of claim 22, wherein said first α/β peptide domain and/or said second α/β peptide domain are each selected from the group consisting of: SEQ ID NO.'s 1-6, 57, or a variant or homolog thereof.

28. The composition of claim 22, wherein said first α/β peptide domain comprises a unit A peptide and said second α/β peptide domain is a C/B peptide, or a unit D peptide.

29. The composition of claim 28, wherein said chimeric peptide sequence is selected from the group consisting of: SEQ ID NO.'s 21-45, and 54, or a fragment or variant thereof.

30. (canceled)

31. The composition of claim 22, wherein said first α/β peptide comprises the peptide having an amino acid sequence according to SEQ ID NO.'s 44-52, or a fragment or variant thereof, and wherein said second α/β peptide comprises the peptide having an amino acid sequence according to SEQ ID NO.'s 53 or 57, or a fragment or variant thereof.

32-34. (canceled)

35. The composition of claim 22, wherein said pathogen comprises a gram-negative bacterial infection caused by: Erwinia amylovora, Candidatus Liberibacte asiaticus (CLas), and Xylella fastidiosa (Xf).

36. The composition of claim 22, wherein said plant is a fruit plant, a citrus plant, an apple plant, a grape plant, or a pear plant.

37. The composition of claim 22, wherein said plant comprises a plant infected with, or at risk of being infected with Huanglongbing (HLB), Fire blight, or Pierce's disease.

38. The composition of claim 22, wherein increasing the innate immune response comprises modulating the expression of one or more citrus genes selected from: Lipid-transfer protein 2, LTP2 (XM_006482145.3), Ethylene-responsive transcription factor 3, ERF003 (XM_006483296.3), Chitinase (XM_015532796.2), Zinc finger, C2H2 type (XM_015531045.2), GDSL esterase (XM_006478917.3), Abscisic acid induced-regulated protein (XM_025101123.1), LEA protein5, LEA5 (NM_001289140.1), Cytochrome P450 82G1 (XM_006479159.3), sodium/hydrogen exchanger 2 (XM_006479811.3), Phloem-specific lectin PP2-like protein (XM_025095878.1), Ethylene-responsive transcription factor 6, ERF006 (XM 006466962.3), Sweet sugar transporter 3 (XM_006490501.3), MAPK6 (XM_025097223.1), Defensein (XM_006470821.3), EDS (XM_006476627.2), Col1 (XM_006486308.3), MAPKK3 (XM_006470193.3), WRKY24 (XM_006468068.3), WRKY4 (XM_006483024.2), PR1 (XM_006474081.3), MYB13 (XM_006479482.2), PR2 (KAH9738797.1), and PR3 (KDO71433.1).

39. The composition of claim 22, wherein increasing the innate immune response comprises modulating the expression of one or more apple genes selected from: Jaz17, JA receptor (MDP0000241358), bHLH, JA induced transcription factor (MDP0000242554), EBP, ethylene induced binding GCC element binding transcription factor (MDP0000241358), AP2/ERF: regulates the biosynthesis of carotenoid by regulating the transcription of PSY, PAL1, SA-inducing PHE ammonia lyase 1 (MDP0000388769) Chalcone and stilbene synthase in flavonoid synthesis (MDP0000168735), ribonuclease-like PR (MDP0000782085), Apple defensin (MDP0000362305), Acidic endochitinase-like protein (MDP0000280265), Intracellular Ras-group-related LRR protein (MDP0000281307), Chlorophyl binding protein PSII LHC (MDP0000708928), The light-harvesting complex, LHC (MDP0000601491), NAD(P)H dehydrogenase (MDP0000509613), Peroxidase super family (MDP243237), Sorbitol dehydrogenase, SDH-GroES-like zinc-binding alcohol dehydrogenase family protein (MDP0000515106), (MDP0000850409), (MDP0000364657), Gibberellic acid stimulated Arabidopsis (GASA) gene (MDP0000201700), Xyloglucan endotransglucosylases/hydrolases, XTH (MDP0000361876), Hydroxyproline-rich glycoprotein family protein (MDP0000248516), Fasciclin-like arabinogalactan-protein 7 (MDC015146.108: 31720-32772), MDP0000297541, proline-rich receptor-like protein kinase (MDP0000511014), MDP0000268505, Nicotianamine synthase-like 4 (MDP0000412490), and Hydroxyproline-rich glycoprotein family protein: (MDP0000248516).

40-52. (canceled)


SEQUENCE LISTING (57 sequences)

SEQ ID NO: 1 - protein, 11 aa, synthetic construct
KKLIKKILKIL

SEQ ID NO: 2 - protein, 11 aa, synthetic construct
KKLFKKILKYL

SEQ ID NO: 3 - protein, 11 aa, synthetic construct
FWQRRIRRWRR

SEQ ID NO: 4 - protein, 11 aa, synthetic construct
FQWQRNIRKVR

SEQ ID NO: 5 - protein, 8 aa, synthetic construct
RRWWRWWR

SEQ ID NO: 6 - protein, 16 aa, synthetic construct
IERSTNLDWYKGPTLL

SEQ ID NO: 7 - protein, 8 aa, synthetic construct
GSGYGSPG

SEQ ID NO: 8 - protein, 5 aa, synthetic construct
GRGSE

SEQ ID NO: 9 - protein, 5 aa, synthetic construct
PYSPG

SEQ ID NO: 10 - protein, 5 aa, synthetic construct
PDSPG

SEQ ID NO: 11 - protein, 6 aa, synthetic construct
GYGSPG

SEQ ID NO: 12 - protein, 4 aa, synthetic construct
GSPG

SEQ ID NO: 13 - protein, 4 aa, synthetic construct
SPLE

SEQ ID NO: 14 - protein, 6 aa, synthetic construct
GSKAHL

SEQ ID NO: 15 - protein, 6 aa, synthetic construct
GSKAHE

SEQ ID NO: 16 - protein, 11 aa, synthetic construct
GSYGSVYGSPG

SEQ ID NO: 17 - protein, 21 aa, synthetic construct
GTCGAGCGCGTATGCAATACG

SEQ ID NO: 18 - protein, 24 aa, synthetic construct
CTACCTTTTTCTACGGGATAACGC

SEQ ID NO: 19 - protein, 17 aa, synthetic construct
CACTGATGGTGCCGTTG

SEQ ID NO: 20 - protein, 18 aa, synthetic construct
CGCCAGGATAGTCGCATA

SEQ ID NO: 21 - protein, 30 aa, synthetic construct
KKLPKEILKILGSGYGSLPKEILKILELKK

SEQ ID NO: 22 - protein, 30 aa, synthetic construct
KKLPEKILEILGSGYKKLPFWQRRIRRWRR

SEQ ID NO: 23 - protein, 30 aa, synthetic construct
KKLIKKILKILGSGYGSPGFWQRRIRRWRR

SEQ ID NO: 24 - protein, 30 aa, synthetic construct
FWQRRIRRWRRGSGYGSPGKKLIKKILKIL

SEQ ID NO: 25 - protein, 30 aa, synthetic construct
KKLPKKILKILGSGYGSPGFWQRRIRRWRR

SEQ ID NO: 26 - protein, 27 aa, synthetic construct
RRWWRWWRGSGYGSPGFWQRRIRRWRR

SEQ ID NO: 27 - protein, 27 aa, synthetic construct
FWQRRIRRWRRGSGYGSPGRRWWRWWR

SEQ ID NO: 28 - protein, 27 aa, synthetic construct
RKPARKVLKILGRGSEFWQKRVRRWRR

SEQ ID NO: 29 - protein, 30 aa, synthetic construct
RLPKAFQWQRRLRRWRRPYSPGRRWWRWWR

SEQ ID NO: 30 - protein, 30 aa, synthetic construct
RLPEAFQWQRRLRRWRRPDSPGRRWWRWWR

SEQ ID NO: 31 - protein, 30 aa, synthetic construct
RLPEAFQWQRNIRKVRRPDSPGRRWWRWWR

SEQ ID NO: 32 - protein, 30 aa, synthetic construct
KKLPEKILKILESGYGSPGFWQRRIRRWRR

SEQ ID NO: 33 - protein, 30 aa, synthetic construct
KKLPEKILKILESLKGSPGFWQRRIRRWRR

SEQ ID NO: 34 - protein, 30 aa, synthetic construct
KKLPQKLLEILKSLKGSPGFWQRRIRRWRR

SEQ ID NO: 35 - protein, 30 aa, synthetic construct
KKLPEKLLEILKSLEGSPGFWQRRIRRWRR

SEQ ID NO: 36 - protein, 30 aa, synthetic construct
KPRGSEQLQELTRRLLDSPLERRWWEWMRR

SEQ ID NO: 37 - protein, 30 aa, synthetic construct
KPRLSEQLQELTRRLLDSPLERRWWEWWRR

SEQ ID NO: 38 - protein, 30 aa, synthetic construct
KPRGSEQLQELTRRLLDSPLERRFWQWMRR

SEQ ID NO: 39 - protein, 28 aa, synthetic construct
KRPEELLQKLKSLEGSKAHLQHHDWTSK

SEQ ID NO: 40 - protein, 30 aa, synthetic construct
LPKRLEELLQKLKSLEGSKAHEKLHDWTRK

SEQ ID NO: 41 - protein, 30 aa, synthetic construct
QPKRLEELLEKLKSLEGSKAHEKLHDWTRK

SEQ ID NO: 42 - protein, 30 aa, synthetic construct
RRWWRWWRGSYGSVYGSPGFWQRRIRRWRR

SEQ ID NO: 43 - protein, 27 aa, synthetic construct
IERSRNLDWYKGPTLLDALKNLNEGKR

SEQ ID NO: 44 - protein, 31 aa, synthetic construct
IERSRNLDWYKGPTLLDALKNLNEGKRPSDK

SEQ ID NO: 45 - protein, 19 aa, synthetic construct
KCRRLCYKQRCVTYCRGRQ

SEQ ID NO: 46 - protein, 13 aa, synthetic construct
KKLPEKILKILES

SEQ ID NO: 47 - protein, 11 aa, synthetic construct
FWQRRIRRWRR

SEQ ID NO: 48 - protein, 15 aa, synthetic construct
KKLPEKILKILESLK

SEQ ID NO: 49 - protein, 11 aa, synthetic construct
FWQRRIRRWRR

SEQ ID NO: 50 - protein, 17 aa, synthetic construct
RLPEAFQWQRNIRKVRR

SEQ ID NO: 51 - protein, 8 aa, synthetic construct
RRWWRWWR

SEQ ID NO: 52 - protein, 11 aa, synthetic construct
KKLPKKILKIL

SEQ ID NO: 53 - protein, 11 aa, synthetic construct
FWQRRIRRWRR

SEQ ID NO: 54 - protein, 31 aa, synthetic construct
KCRRLCYKQRCVTYCRGRQPEKILKILESLK

SEQ ID NO: 55 - protein, 20 aa, synthetic construct
AAGGCAATAAACGCGCACTA

SEQ ID NO: 56 - protein, 20 aa, synthetic construct
GGTTTTGCTGACTGGCAACA

SEQ ID NO: 57 - protein, 14 aa, synthetic construct
RQPEKILKILESLK